SEQUENCE LISTING (23 sequences)

SEQ ID NO: 1 - DNA, 20 bp, Artificial Sequence
  Oligonucleotide
aattggaggagggaaggggg

SEQ ID NO: 2 - DNA, 20 bp, Artificial Sequence
  Oligonucleotide
catgggaggagggaaggggg

SEQ ID NO: 3 - DNA, 16 bp, Artificial Sequence
  Oligonucleotide
cccccttccctcctcc

SEQ ID NO: 4 - DNA, 16 bp, Artificial Sequence
  Oligonucleotide
gggctggagagatggc

SEQ ID NO: 5 - DNA, 16 bp, Artificial Sequence
  Oligonucleotide
gagatattaattggct

SEQ ID NO: 6 - DNA, 16 bp, Artificial Sequence
  Oligonucleotide
gccaatggctgggcag

SEQ ID NO: 7 - DNA, 16 bp, Artificial Sequence
  Oligonucleotide
gaaatgcaaatcaaaa

SEQ ID NO: 8 - DNA, 16 bp, Artificial Sequence
  Oligonucleotide
tggcgaggatgtggag

SEQ ID NO: 9 - DNA, 16 bp, Artificial Sequence
  Oligonucleotide
gctccactatgttcat

SEQ ID NO: 10 - DNA, 16 bp, Artificial Sequence
  Oligonucleotide
cagtattgttttatta

SEQ ID NO: 11 - DNA, 16 bp, Artificial Sequence
  Oligonucleotide
gaaacttagtctcctg

SEQ ID NO: 12 - DNA, 16 bp, Artificial Sequence
  Oligonucleotide
cattatatggatgata

SEQ ID NO: 13 - DNA, 16 bp, Artificial Sequence
  Oligonucleotide
agttcgaggccagcct

SEQ ID NO: 14 - DNA, 16 bp, Artificial Sequence
  Oligonucleotide
gagttcgaggccagcc

SEQ ID NO: 15 - DNA, 16 bp, Artificial Sequence
  Oligonucleotide
ccagcactcgggaggc

SEQ ID NO: 16 - DNA, 25 bp, Artificial Sequence
  Oligonucleotide
agtggacgtggacagaattcggatc

SEQ ID NO: 17 - DNA, 26 bp, Artificial Sequence
  Oligonucleotide
tcaccctgcacctgtcttaagcctag

SEQ ID NO: 18 - DNA, 16 bp, Artificial Sequence
  Oligonucleotide
agtgggacgtggacag

SEQ ID NO: 19 - DNA, 20 bp, Artificial Sequence
  Oligonucleotide
tcaccctgcaccngncttaa

SEQ ID NO: 20 - DNA, 35 bp, Artificial Sequence
  Oligonucleotide
gtgggacgtggacagaattggaggagggaaggggg

SEQ ID NO: 21 - DNA, 16 bp, Artificial Sequence
  Oligonucleotide
cctcctcccttccccc

SEQ ID NO: 22 - DNA, 20 bp, Artificial Sequence
  Oligonucleotide
aattggaggagggaaggggg

SEQ ID NO: 23 - DNA, 16 bp, Artificial Sequence
  Oligonucleotide
gatccgaattctgtcc

(19) United States
(12) Patent Application Publication
(10) Pub. No.: US 2010/0304990 A1
(43) Pub. Date: Dec. 2, 2010

(54) RULER ARRAYS

(76) Inventors: David K. Gifford, Weston, MA (US); P. Alexander Rolfe, Cambridge, MA (US)

Correspondence Address: WOLF GREENFIELD & SACKS, P.C., 600 ATLANTIC AVENUE, BOSTON, MA, 02210-2206, US

(21) Appl. No.: 12/668,944

(22) PCT Filed: Jul. 16, 2008
(86) PCT No.: PCT/US08/08743

§ 371 Date: Aug. 19, 2010

Related U.S. Application Data
(60) Provisional application No. 60/959,791, filed on Jul. 16, 2007.
(60) Provisional application No. 60/959,834, filed on Jul. 17, 2007.

(51) Int. Cl. C12Q 1/68; C40B 30/04; C07H 21/04; C40B 50/06

(52) U.S. Cl. 506/9; 435/6; 536/23.1; 506/26

(57) ABSTRACT

The invention in some aspects relates to methods for measuring distances between locations in a nucleic acid. The invention relates to methods of genetic analysis useful for detecting genomic alterations. In some aspects, the invention relates to methods for detecting genomic insertions, deletions, and inversions.

### RELATED APPLICATIONS

This application claims the benefit under 35 U.S.C. §119(e) of the filing date of U.S. Provisional Application Ser. No. 60/959,791 (Attorney Docket No.: M0656.70142US00) filed Jul. 16, 2007 and U.S. Provisional Application Ser. No. 60/959,834 (Attorney Docket No.: M0656.70142US01) filed Jul. 17, 2007. The entire teachings of the referenced provisional applications are expressly incorporated herein by reference.

### GOVERNMENT FUNDING

This invention was made with Government support from the National Institutes of Health under Grant No. 5-R01-GM069676-02. The United States Government has certain rights in the invention.

### FIELD OF THE INVENTION

The invention in some aspects relates to methods for measuring distances between two or more locations in a nucleic acid. The invention relates to methods of genetic analysis useful for detecting genomic alterations. In some aspects, the invention relates to methods for detecting genomic insertions, deletions, and inversions.

### BACKGROUND OF THE INVENTION

Analyzing genome composition and organization is important to understanding the genetics of human development and disease. Fueled by the sequencing of the human genome, quantitative genetic analysis is a rapidly evolving field that has been supported by the development of many new analytical methods. A common characteristic of presently-available methods of such analysis is that they lack the ability to detect certain changes in genome features, such deletions, insertions and inversions, in a systematic and efficient way.

### SUMMARY OF THE INVENTION

The invention relates to a method of genetic analysis useful for detecting insertions, deletions, and inversions between a nucleic acid and a reference genome or between two nucleic acids. The invention entails producing a collection of nucleic acid fragments wherein the frequency of occurrence of fragments of a given length relates to that length. In one embodiment, DNA polymerase molecules begin extensions at a defined set of points in an input nucleic acid, which is a nucleic acid to be assessed. Extension terminates at each base (either naturally or by incorporating a ddNTP molecule) and, thus, long extension products are less likely to be produced than short products. Therefore, a probe that queries for an extension product close to a defined initiation point will yield a stronger signal than a probe that queries for a product farther from the defined initiation point. A resulting hybridization pattern, which is a set of probe signals, can be compared either (1) to a hybridization pattern predicted from a reference sequence or (2) to a hybridization pattern produced by a reference nucleic acid. In either case, differences between hybridization patterns indicate that one or more of the query probes has changed its distance from an initiation point in the sample.

In some aspects, the invention provides a method for measuring the distance between locations in a nucleic acid (a nucleic acid to be assessed), wherein the locations are a predefined location and a test location. The method comprises: (a) preparing nucleic acid fragments from the nucleic acid, wherein each fragment comprises (i) only one predefined region, wherein the predefined region is complementary to a predefined location of the nucleic acid and (ii) at least one test region, wherein a test region is complementary to a test location of the nucleic acid, and (b) measuring the frequency of occurrence of each test region in the nucleic acid fragments, wherein the frequency of occurrence of a particular test region is inversely related to the distance between the test location in the nucleic acid that is complementary to the particular test region and the predefined location in the nucleic acid.

In some embodiments, the measuring comprises: contacting nucleic acid fragments prepared in (a) with at least one polynucleotide under conditions appropriate for hybridization of nucleic acid fragments (hybridization of polynucleotides), wherein each polynucleotide is complementary to a test region, and assessing hybridization of nucleic acid fragments with the at least one polynucleotide, wherein the extent of hybridization is indicative of the frequency of occurrence of the test region complementary to the at least one polynucleotide.

In some embodiments, the measuring comprises sequencing the nucleic acid fragments prepared in (a) to obtain fragment sequences and assessing the occurrence of each test region in the fragment sequences to obtain the frequency of occurrence of each test region in the nucleic acid fragments.

In some embodiments, the predefined location is a restriction site.

In some embodiments, the preparing comprises digesting the nucleic acid with a restriction enzyme at the restriction site to produce restriction fragments.

In some embodiments, the methods involve ligating an adapter to the restriction fragment ends to produce adapter ligated restriction fragments.

In some embodiments, the methods involve performing a extension reaction on the adapter ligated restriction fragments to produce the nucleic acid fragments, wherein the reaction includes a polymerase, a primer complementary to the adapter, a reaction buffer, and a nucleotide mixture.

In some embodiments, the preparing comprises performing a extension reaction on the nucleic acid to produce the nucleic acid fragments, wherein the reaction includes a polymerase, a primer complementary the predefined location, a reaction buffer, and a nucleotide mixture.

In some embodiments, the nucleotide mixture comprises one or more dideoxynucleotides.

In some embodiments, the nucleotide mixture comprises one or more labeled nucleotides.

In some embodiments, the labeled nucleotides are Cy5-dUTP, Cy3-dUTP, or amine modified nucleotides

In some embodiments, the methods involve conjugating labels to the amine modified nucleotides after the extension reaction.

In some embodiments, the methods involve separating labeled nucleic acid fragments.

In some embodiments, the preparing comprises incorporating a biotin moiety in nucleic acid fragments.

In some embodiments, the nucleic acid fragments are separated by contacting the biotin moiety with streptavidin that is fixed to a solid support under conditions that result in binding of biotin moieties to the streptavidin.

In some embodiments, the preparing comprises sonicating the nucleic acid.

In some embodiments, the methods involve labeling the nucleic acid fragments with a universal labeling system (ULS).

In some embodiments, the at least one polynucleotide is fixed to a solid support.

In some embodiments, the at least one polynucleotide is a constituent of a query probe.

In some embodiments, the solid support is an array.

In some embodiments, the array is a genome microarray, chromosome array, or CpG island array.

In some embodiments, the nucleic acid is RNA or DNA.

In some embodiments, the nucleic acid is a genome.

In some aspects, the invention provides methods for detecting an aberration in a nucleic acid. The methods involve determining a distance between locations in the nucleic acid by any of the foregoing methods, and comparing the distance to a reference distance wherein the result of the comparison is indicative of the aberration. If the distance between two locations is different in a nucleic acid from a reference distance (e.g., the distance between the two locations in a corresponding wild-type or non-aberrant nucleic acid), there is an aberration in the nucleic acid.

In some embodiments, the aberration is an inversion, insertion, or deletion.

In some aspects, the invention relates to a method for detecting a difference between a test nucleic acid and a reference nucleic acid, wherein the method comprises: (a) contacting (i) a collection of labeled test nucleic acid fragments with (ii) a set of query probes, wherein test nucleic acid fragments are labeled at one or more defined sites, to produce labeled test nucleic acid fragments and wherein a query probe is a polynucleotide and the set of query probes comprises at least three different polynucleotides, each of whose sequence identifies a known region in the reference nucleic acid, under conditions appropriate for hybridization of labeled test nucleic acid fragments with query probes; (b) determining the extent of hybridization between each query probe and labeled test nucleic acid fragments; (c) associating the extent of hybridization for each query probe, characteristic(s) of the known region identified by the query probe, and characteristic(s) of the defined sites, to produce a test hybridization pattern; (d) determining distance in the test hybridization pattern by evaluating the extent of hybridization for a query probe within the resolution limit of a defined site within the test hybridization pattern with (i) the extent of hybridization of the query probe in a reference hybridization pattern and (ii) distance from the query probe to the defined site in the reference hybridization pattern, wherein distance is the number of bases between a defined site and a region identified by a query probe; and (e) identifying a difference in distance between the reference hybridization pattern and the test hybridization pattern, thereby detecting a difference between a test nucleic acid and a reference nucleic acid.

In some aspects, the invention relates to a method for detecting a difference between a test nucleic acid and a reference nucleic acid, wherein the method comprises: (a) contacting (i) a collection of labeled test nucleic acid fragments with (ii) a set of query probes, wherein test nucleic acid fragments are labeled at one or more defined sites, to produce the labeled test nucleic acid fragments and wherein a query probe is a polynucleotide and the set of query probes comprises at least three different polynucleotides, each of whose sequence identifies a known region in the reference nucleic acid, under conditions appropriate for hybridization of labeled test nucleic acid fragments with query probes; (b) determining the extent of hybridization between each query probe and labeled test nucleic acid fragments; (c) associating the extent of hybridization for each query probe, characteristic(s) of the known region identified by the query probe, and characteristic(s) of the defined sites, to produce a test hybridization pattern; (d) comparing the test hybridization pattern with a reference hybridization pattern to produce a ratio hybridization pattern; and (e) identifying a significant local maximum or a significant local minimum in the ratio hybridization pattern, thereby detecting a difference between a test nucleic acid and a reference nucleic acid.

In some embodiments of the foregoing methods, the lengths of the test and reference nucleic acid fragments have a random distribution. In certain embodiments, the random distribution of test nucleic acid fragments is substantially equivalent to the random distribution of reference nucleic acid fragments. In other embodiments, the majority of fragments are from about 3-kb to about 5-kb.

In some embodiments of the foregoing methods, the defined sites are defined by the sequence specificity of one or more restriction enzymes. In certain embodiments, one of the one or more restriction enzymes is EcoRI. In certain other embodiments, one of the one or more restriction enzymes is BamHI. In certain other embodiments, at least one of the one or more restriction enzymes is methylation sensitive. In certain other embodiments, the method further comprises contacting labeled nucleic acid fragments with the one or more restriction enzymes under conditions suitable for digestion of the nucleic acid fragments by the one or more restriction sites at defined sites, thereby producing digested labeled nucleic acid fragments. In certain other embodiments, the method further comprises ligating an adapter to digested nucleic acid fragments to produce linker-ligated nucleic acid fragments. In certain other embodiments, the adapter comprises at least one detectable nucleotide. In certain other embodiments, the method further comprises linear PCR in which the linker-ligated nucleic acid fragments serve as a template to produce the labeled nucleic acid fragments. The linear PCR is primed by a primer comprising a sequence complementary to a portion of the linker.

In some embodiments of the foregoing methods, the defined sites are specified by one or more PCR primers, wherein the PCR primers are used to prime a linear PCR reaction with the nucleic acid fragments as a template. In certain embodiments, the linear PCR incorporates a detectable nucleotide, thereby producing the labeled nucleic acid fragments. In specific embodiments, the detectable nucleotide is a fluorophore-conjugated nucleotide. In other embodiments, the fluorophore has an excitation peak of about 492 nm and emission peak of about 510 nm, an excitation peak of about 550 nm and emission peak of about 570 nm, or an excitation peak of about 650 nm and emission peak of about 670 nm. In further embodiments, the fluorophore is Cy3 or Cy5.

In some embodiments of the foregoing methods, the query probes are arranged in an array. In specific embodiments, the array is a genomic microarray, a chromosome array, or a CpG island array.

Further embodiments of the invention relate to methods for labeling DNA, wherein the methods comprise: (a) combining: (i) linear DNA that comprises DNA to be labeled and, adapter DNA that tags each end of the DNA to be labeled, wherein the adapter DNA flanks the DNA to be labeled; (ii) primer capable of hybridizing to the adapter DNA; and (iii) labeled nucleotides or combining: (i) linear DNA to be labeled (ii) a primer capable of hybridizing to a specific sequence in the linear DNA; and (iii) labeled nucleotides, thereby producing a combination; and (b) maintaining the combination under conditions appropriate for amplification of the linear DNA to occur, thereby producing amplified DNA comprising at least one labeled nucleotide, thereby producing labeled DNA.

A further embodiment of the invention relates to methods for producing a pool of labeled DNA fragments, wherein the pool comprises a random distribution of labeled DNA fragments of from about 3 kilobases to about 5 kilobases, wherein the methods comprise: (a) combining: (i) linear DNA that comprises DNA to be labeled and, adapter DNA that tags each end of the DNA to be labeled, wherein the adapter DNA flanks the DNA to be labeled; (ii) primer capable of hybridizing to the adapter DNA; and (iii) labeled nucleotides or combining: (i) linear DNA to be labeled; (ii) a primer capable of hybridizing to a specific sequence in the linear DNA; and (iii) labeled nucleotides, thereby producing a combination; and (b) maintaining the combination under conditions appropriate for amplification of the linear DNA to occur, thereby producing amplified DNA comprising at least one labeled nucleotide, thereby producing a pool of labeled DNA fragments.

In some aspects the invention provides methods for detecting insertions and deletions between a test nucleic acid and a reference sequence.

In some embodiments, the methods for detecting insertions and deletions involve (a) generating a collection of labeled nucleic acid fragments, wherein each fragment originates at one of a set of defined locations in the nucleic acid and wherein the number of fragments terminating at some location (a particular location) in the nucleic acid is related to that location's distance from an originating site; (b) contacting the labeled test nucleic acid fragments with a set of query probes, wherein each query probe is a polynucleotide and the set of query probes comprises at least three polynucleotides, each of whose sequence identifies a known region in the reference sequence, under conditions appropriate for hybridization of labeled test nucleic acid fragments with query probes; (c) determining the extent of hybridization between each query probe and the labeled nucleic acid fragments; (d) associating the extent of hybridization for each query probe with the location in the reference sequence against which the probe was designed to produce a test hybridization pattern; (e) determining distances between probes and other points (positions) in the test hybridization pattern; and (f) determining the locations of insertions and deletions between the test nucleic acid and the reference sequence by comparing the observed pattern of hybridization to the hybridization pattern that one would predict from the reference sequence.

In some embodiments, the methods for detecting insertions and deletions involve (a) generating two collections of labeled nucleic acid fragments from different nucleic acids, wherein each fragment originates at one of a set of defined locations in the nucleic acid and the number of fragments terminating at some location in the nucleic acid is related to that location's distance from an originating site; (b) contacting the two collections of differently labeled test nucleic acid fragments with a set of query probes wherein each query probe is a polynucleotide and the set of query probes comprises at least three polynucleotides, each of whose sequence identifies a known region in the reference sequence, under conditions appropriate for hybridization of labeled test nucleic acid fragments with query probes; (c) determining the extent of hybridization between each query probe and each of the labeled nucleic acid fragments (d) associating the extent of hybridization for each query probe for each test nucleic acid with the location in the reference sequence against which the probe was designed to produce a test hybridization pattern; (e) determining distances between probes and other points in the test hybridization pattern; and (f) determining the locations of insertions and deletions between the test nucleic acid and the reference sequence.

In some embodiments, the origins (defined locations) of the collection or collections of nucleic acid fragments are defined by a set of locations in the test nucleic acid(s) cleaved by a (one or more) restriction enzyme(s).

In some embodiments, each template nucleic acid is digested by a restriction enzyme and an adapter molecule is ligated primarily to the nucleic acid ends resulting from the digesting. In certain embodiments, a primer complementary to the adapter is used to initiate an extension reaction by a DNA polymerase at the restriction sites.

In some embodiments, the origins (defined locations) of the collection or collections of nucleic acid fragments are defined by a (one or more) nicking DNA endonuclease(s) that nick the template nucleic acid (test nucleic acid) to allow a DNA polymerase to begin synthesis at the nick.

In some embodiments, the origins (defined locations) of the collection or collections of nucleic acid fragments are defined by a (one or more) single-stranded oligonucleotide primer(s) that is (are) complementary the template nucleic acid at least one position, wherein the origin(s) is (are) the site(s) of complementarity in the template nucleic acid.

In some embodiments, the lengths of the labeled nucleic acid fragments are determined by sonicating the nucleic acid prior to generating the labeled fragments.

In some embodiments, the length of the labeled nucleic acid fragments are determined by the processivity of a DNA polymerase that began synthesis of a labeled fragment at one of the defined sites and terminated synthesis randomly.

In some embodiments, the lengths of the labeled nucleic acid fragments are determined by the concentration of ddNTPs in the reaction that produced the labeled nucleic acid fragments where a DNA polymerase began synthesis of a labeled fragment at one of the defined sites in the input nucleic acid and terminated synthesis upon incorporating a ddNTP.

In some embodiments, the labeled nucleic acid fragments are produced by a DNA polymerase incorporating dye-conjugated dNTP molecules in addition to unlabeled dNTPs as it synthesizes the fragment from one of the defined sites in the input nucleic acid.

In some embodiments, the labeled dNTP molecules are conjugated to a dye having an excitation peak of about 492 nm and emission peak of about 510 nm, an excitation peak of about 550 nm and emission peak of about 570 nm, or an excitation peak of about 650 nm and emission peak of about 670 nm

In some embodiments, the labeled dNTP molecules are Cy5-dUTP or Cy3-dUTP.

In some embodiments, the labeled dNTP is amine modified, but does not carry a fluorophore, and a dye is attached to an extension product after an extension reaction.

In some embodiments, the labeled nucleic acid fragments are separated from the template nucleic acid to prevent the template nucleic acid material from interfering with the hybridization of the labeled nucleic acid fragments with the query probes.

In some embodiments, the template nucleic acid molecules typically contain one or more biotin molecules and are extracted from the reaction with streptavidin beads to leave behind primarily the labeled nucleic acid fragments.

In some embodiments, the adapter molecule contains one or more chemical modifications or attachments to permit separation of (1) the successfully ligated template nucleic acid from the remainder of the input nucleic acid (nucleic acid to be assessed) and (2) the separation of the labeled nucleic acid product from the template nucleic acid. In certain embodiments, the adapter molecule contains one or more detectable nucleotides, with the result that the linker-ligated fragment is labeled. In certain embodiments, the adapter molecule contains one or more biotin molecules to permit purification using streptavidin beads.

In some embodiments, unlabeled dNTPs are incorporated by the polymerase and the resulting product is labeled after purification from the template.

In some embodiments, the labeling is by the Universal Linkage System (ULS) (See van Gijlswijk R P, et al., Expert Rev Mol Diagn. 2001 May; 1(1):81-91).

In some embodiments, the labeling is performed by amine modification followed by labeling, for example, with succinimidyl ester dyes.

In some embodiments, the query probes are arranged on a array. In certain embodiments, the array is a microarray, a genomic microarray, chromosome array, or a CpG island array. In particular embodiments, the array contains query probes in the specific genomic loci of interest to the experimenter.

In some embodiments, the distribution of labeled nucleic acid fragment lengths is exponential or roughly (approximately) exponential such that the log intensities observed by the query probes can be modeled as a line.

### BRIEF DESCRIPTION OF THE DRAWINGS

FIG. 1 depicts that a ruler array relies on probabilistic breaking, also referred to as the random fragmentation, of genomic DNA such that as the two ends of the ruler move farther apart in the genome, the probability of a DNA fragment containing both ends decreases. Imagine fixing a label to some point in the genome and randomly fragmenting many copies of that genome. When the resulting material hybridizes to a microarray, probes near the labeled site will show higher intensities than probes farther away because fewer breaks occur over a short distance than a long one. The fraction of the genome interrogated by this method depends on the distribution of labeled sites throughout the genome, the length of DNA fragments, and the presence of microarray probes in the genome. Several methods could suitably break the genomic DNA. While sonication or pipetting would break the DNA randomly or pseudorandomly, incomplete restriction enzyme digestion would probabilistically cut the DNA at certain locations. Probabilistic “falling off” of DNA polymerase during a label incorporation reaction may also be used.

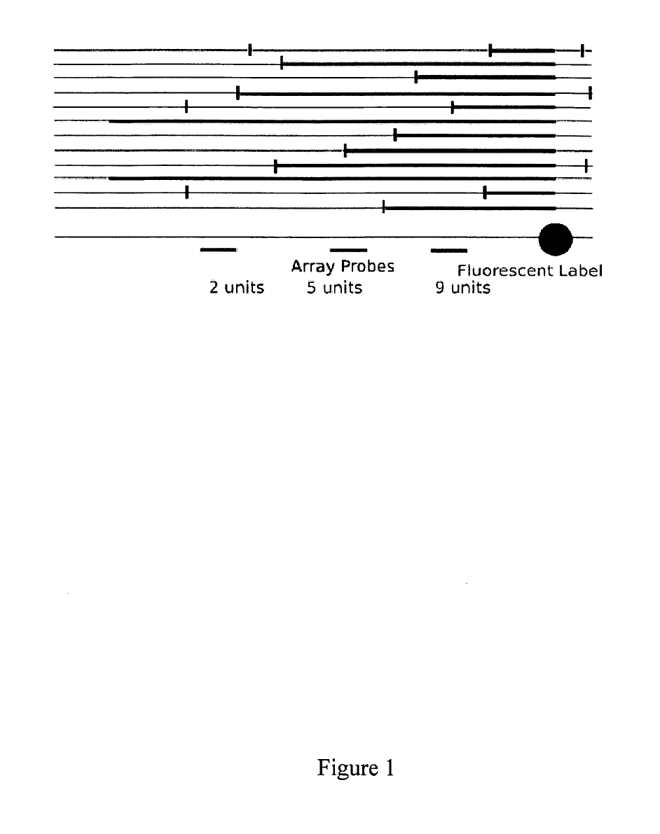

FIG. 2 depicts that array probes complementary to the material produced by the labeled site will show high intensity close to the site and lower intensity at longer distances. At some distance, the observed probe intensities will fall to a background level; the maximum length of DNA fragments and the limitations of the labeling technique determine this distance.

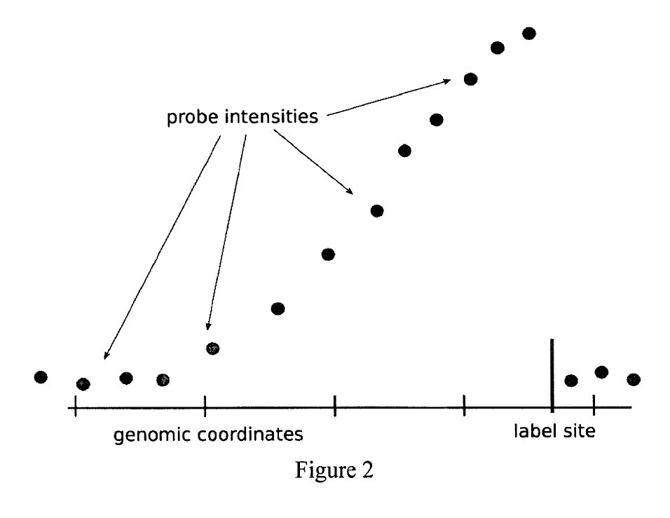

FIG. 3 depicts that when the distance between a probe and a labeling site increases compared to the expected distance, the probes will observe lower intensities than expected. It is possible to determine the location of an insertion by observing a more rapid decrease in intensity than the expected distances alone would predict.

FIG. 4 depict that large deletions will cause some probes to yield extremely low values as the genomic sequence complementary to the probe is not present in the sample. Probes farther from the label site than the deletion will produce higher than expected intensities. Small deletions may not delete any probes from the genome, but will still produce higher than expected intensities at probes beyond the insertion.

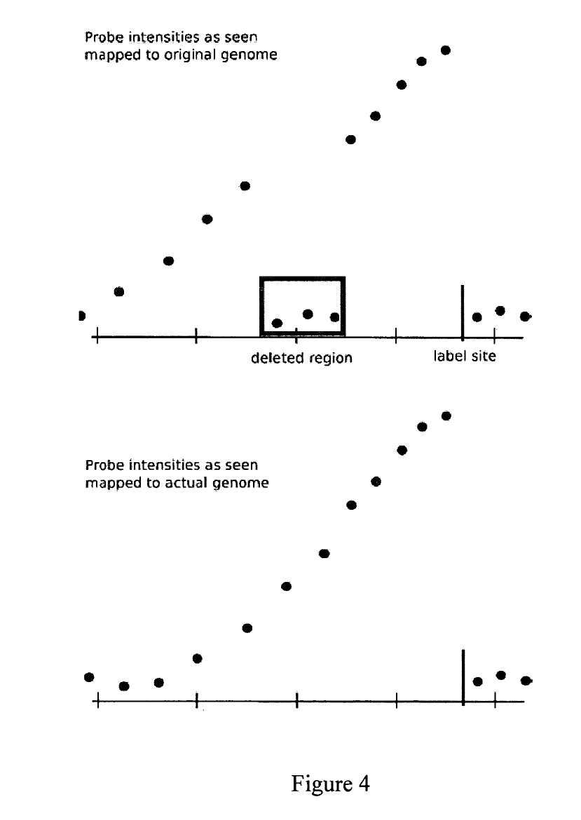

FIG. 5 depicts a procedure for estimating the size of an insertion as the amount of DNA that best matches the observed decrease in probe intensity.

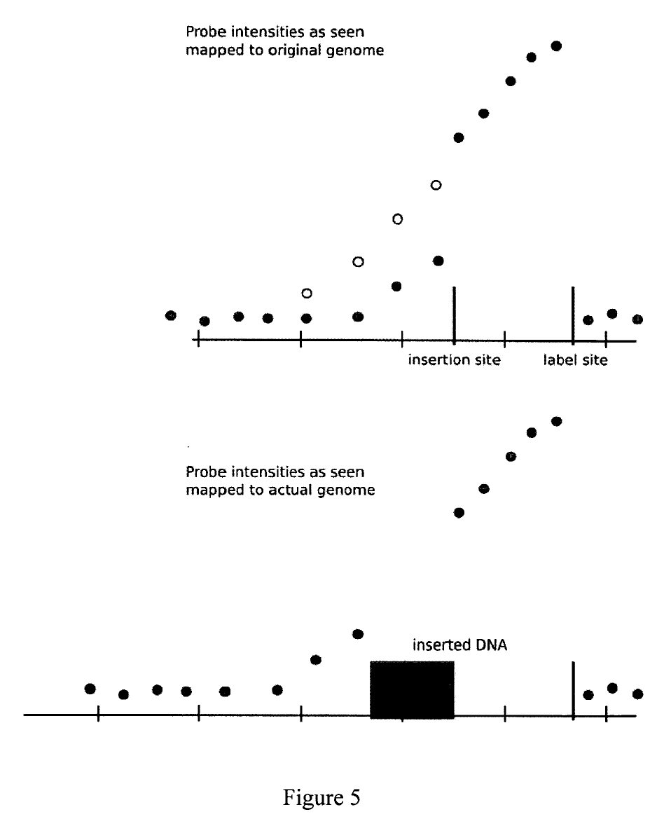

FIG. 6 depicts that an inverted segment of DNA is observable because the pattern of observed probe intensities does not match the expected pattern.

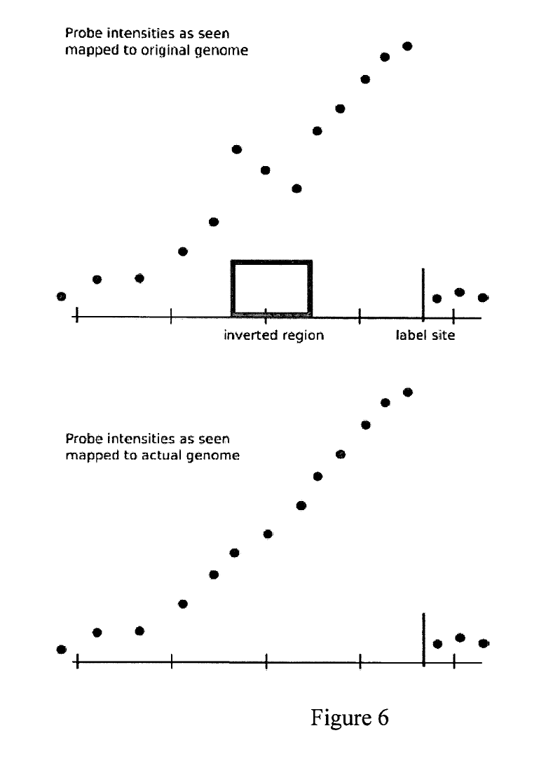

FIG. 7 depicts that probes between an insertion/deletion (indel) and the label site will yield a ratio of roughly one since these probes are the same distance from the label site in both samples. Probes beyond the indel site will yield ratios significantly above or below one since the intensities in one channel will be higher than the intensities in the channel whose probes are now farther away.

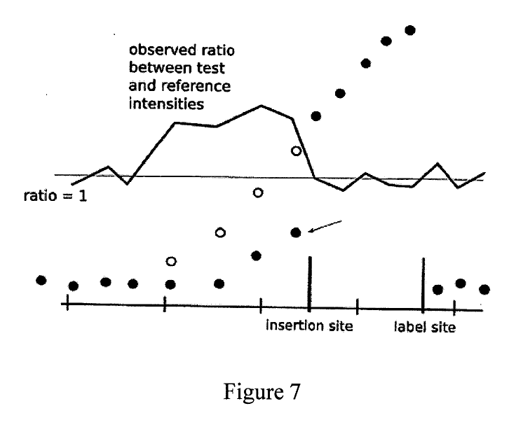

FIG. 8 is a schematic of the distance analysis described in Example 2.

FIG. 9 depicts a method for purifying ligated material on streptavidin beads and then extending from the adapter to product a range of fragment lengths.

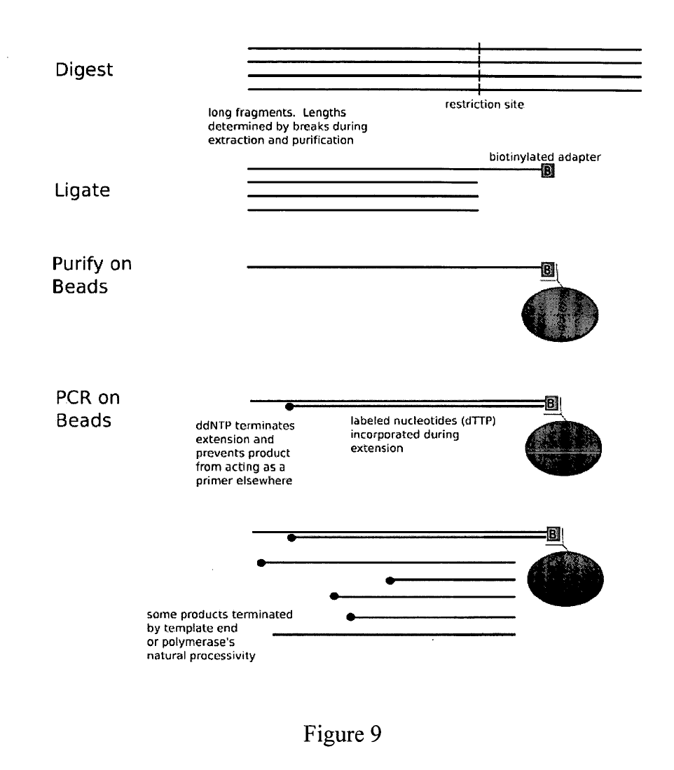

FIG. 10 depicts results of an algorithm that fits observations in an interval of hybridization intensities to either a single line segment or two line segments.

### DETAILED DESCRIPTION OF THE INVENTION

The invention in some aspects relates to methods for measuring distances between two or more locations in a nucleic acid. The ability to measure distances between locations in nucleic acids provides a novel way for interpreting and monitoring genome plasticity, which is crucial to understanding the process of evolution, adaptation, and genetic disease. In some aspects, the methods are useful for efficient and accurate measurement of genome plasticity. The methods are useful for assessing genome plasticity of prokaryotic and eukaryotic cells. Genome plasticity refers to the propensity of a genome to be altered. Such genomic alterations may be deletions, insertions, inversion, translocations, or other rearrangements that include, for example, single nucleotide polymorphisms. Consequently, in some aspects, the invention relates to methods for detecting genomic alterations such as insertions, deletions, and inversions. The methods can be employed to assess genome plasticity in human development and disease, such as cancer. In some aspects, the methods of the invention, are useful for assessing the quality of genome sequencing. For example, sequencing through repetitive elements can be difficult and lead to erroneous results such as improper estimates of repetitive element lengths. In some aspects the invention provides methods of genetic analysis useful for detecting genomic alterations in repetitive elements based on distances between genomic locations. For example, the methods are useful for detecting changes in telomeric proximal regions, repetitive DNA elements, such as, LINE, SINE, Retroviral Sequences, Transposable Elements, Pseudogenes, Ribosomal Genes, Intergenic Tandem Repeats, CAG repeats, and other repetitive elements known to one of ordinary skill in the art.

Nucleic acids are polymers of nucleotides (e.g., deoxynucleotides, ribonucleotides) and may be naturally occurring or non-naturally occurring. They may be harvested from naturally occurring sources or they may be synthetic and prepared by for example nucleic acid synthesizers. Nucleic acids include DNA and RNA, including genomic DNA (e.g., nuclear DNA or mitochondrial DNA), cDNA (or reverse transcript mRNA), mRNA, miRNA, pre-mRNA, artificial chromosomes (e.g., BAC or YAC), cosmid DNA, plasmid DNA, and phagemid DNA. Nucleic acids may be single stranded or double stranded, and may have blunt ends or overhangs. A nucleic acid may be a genome consisting of more than one chromosome. In some embodiments, the methods are used to detect differences is distances in RNA, typically pre-messenger RNA and/or messenger RNAs. In one embodiment, differences in distance between two or more mRNA transcripts are related to differences in RNA processing.

A test nucleic acid is any nucleic acid to be analyzed, such as for genome organization (e.g., a nucleic acid whose organization is not completely known prior to analysis). A reference nucleic acid is, for example, a nucleic acid for which genome organization (total or partial) is known, and against which a set of query probes has been defined. In one embodiment, a test nucleic acid is examined using a set of query probes that specify, by sequence complementarity, positions on the reference nucleic acid. In one embodiment test and reference nucleic acids are genomic DNA.

Nucleic acids can be from any appropriate source including but not limited to nucleic acid from any organism (e.g., human or nonhuman, e.g., bacterium, virus, yeast, fungus, plant, protozoan), nucleic acid-containing samples of tissues, bodily fluids (for example, blood, serum, plasma, saliva, urine, tears, semen, vaginal secretions, lymph fluid, cerebrospinal fluid or mucosa secretions), fecal matter, individual cells or extracts thereof that contain nucleic acid, and subcellular structures such as mitochondria or chloroplasts. Nucleic acid can also be obtained from forensic, food, archeological, or inorganic samples onto which nucleic acid has been deposited or from which it can be extracted. In one embodiment, the nucleic acid has been obtained from a human or animal to be screened for the presence of one or more genetic alterations that can be diagnostic for, or predispose the subject to, a medical condition or disease. Target nucleic acids may be harvested from such sources using the method described herein or by known techniques in the art. See for example Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981).

In one embodiment, a method of measuring the distance between two locations in a nucleic acid (an input nucleic acid). Locations, which may be predefined locations or test locations, are one or more consecutive nucleic acid residues (e.g., a nucleic acid sequence). In some embodiments, a location is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more consecutive nucleic acids. In some embodiments, a location is about 10, about 100, about 1000, about 10000 or more nucleic acid residues. Distances between locations may be measured from any nucleic acid residue within the location (e.g., the first residue). Distance may be an absolute distance (e.g., nucleotide number) or may be a relative distance (e.g., difference in distance).

Methods for measuring distances between two locations in a nucleic acid involve preparing nucleic acid fragments from a nucleic acid (also, referred to as template nucleic acid) in which the distance between two locations is to be determined. The nucleic acid fragments provide information about distances between locations in the template nucleic acid. The template nucleic acid is fragmented to produce a pool (collection) of nucleic acid fragments having a random distribution of sizes. Nucleic acid fragments comprise a predefined region (present at one end), corresponding to a predefined location in the template nucleic acid and a region (which can be present at the second end) corresponding to a random location of the nucleic acid. As used herein, the terms “defined” and “predefined” (e.g., defined locations and predefined locations) are used interchangeably. A predefined region is a region in a nucleic acid fragment from which the distance to a second region in the nucleic acid fragment (a test region) is to be determined/measured. Nucleic acid fragments comprise one or more test regions, each of which has a sequence complementary to a test location of the nucleic acid (the template nucleic acid). Test locations are locations within a nucleic acid whose distance from a predefined location is to be determined. A test region can be at the end of a nucleic acid fragment or can be internal (within the fragment). In some embodiments, a nucleic acid fragment comprises in the following order: a predefined region; a intervening sequence that is not a test region; a test region; and additional sequence that is not a test region. In some embodiments, a nucleic acid fragment comprises in the following order: a predefined region; intervening sequence that is not a test region; and a test region. In some embodiments, nucleic acid fragments comprise multiple distinct (different) test regions separated by intervening sequence that is not a test region.

Test regions can be a variety of sizes. In some embodiments, test regions are about 10, about 20, about 30, about 40, about 50, or about 60 nucleotides in length. In some embodiments, test regions are 25 nucleotides in length. In other embodiments, test regions are 60 nucleotides in length. Test regions are typically selected such that they correspond to only one test location sequence in a template nucleic acid.

Nucleic acids fragments may be a range of sizes. For example, a nucleic acid fragment may be about 10 bp, about 100 bp, about 1 kb, about 10 kb, about 100 kb or more in size. Pools of nucleic acid fragments have a distribution of sizes, and the distance between any position (e.g., a test region) of a nucleic acid fragment and a predefined region of the fragment is inversely related to the frequency of occurrence of the position (e.g., the test region) in the distribution. Inversely related indicates that in a pool of nucleic acid fragments, the greater the distance of a position (e.g., test region) of a nucleic acid fragment from a predefined region (the further away a test position is from the predefined region), the lower the frequency of occurrence of the position in the pool of nucleic acid fragments. Alternatively, the shorter the distance of a position (e.g., test region) from a predefined region (the closer the position is to a predefined region) the greater the frequency of occurrence of the position in the pool of nucleic acid fragments. This is the case because after a nucleic acid is randomly fragmented, the number of nucleic acid fragments that contain any two unique sequences will be inversely proportional to the distance between the two sequences (e.g., test locations and predefined locations). When the sequences are close together, it is likely that fragmenting will not disassociate them and there will be a large number of nucleic acids with both sequences. When the two sequences are far apart, fragmenting is likely to disassociate them, and there will be a correspondingly small number of fragments.

The invention, in some aspects, is based on the discovery that the a pool nucleic acid fragments can be used to infer distances between locations (e.g., predefined locations, test locations) in the nucleic acid from which the fragment were prepared. The pool of nucleic acid fragments consists of a distribution of nucleic acid fragment sizes. The distribution is a set of frequencies of occurrences of nucleic acid fragments of particular sizes present in the pool of nucleic acids fragments. For example, a distribution of fragments produced from a sample of nucleic acid which is genomic DNA may be fragmented to distribution having an average size of about 10 bp, about 100 bp, about 1 kb, about 10 kb, about 100 kb or more. Methods of determining the distribution of sizes are well known in the art. For example, nucleic acids fragments can be resolved by gel electrophoresis (e.g., by agarose gel electrophoresis), stained with a nucleic acid dye (e.g., Ethidium Bromide), and imaged to obtain the fragment size distribution. Nucleic acids may also be resolved by capillary based methods to determine size distributions. The distribution can be characterized in any one of a number of ways known in the art. For example, a mathematical function describing the distribution can be established to relate frequency of occurrence to distance. Theoretical distributions that relate frequency of occurrence to distance may also be determined (See Example 10). These and other methods will be known to the skilled artisan.

Nucleic acids fragments have one or more test regions. Consequently, the distribution of nucleic acid fragment sizes can be related to the set of frequencies of occurrences of particular test regions. Observed occurrences of test regions, for example from a nucleic acid of unknown or partially known structure, may be compared to expected occurrences, for example from a nucleic acid of known structure, to establish relative distances. Frequencies of occurrences of test regions observed in a reference nucleic acid, for which distances between test regions and predetermined regions are known, may be used to establish reference distances or a distance standard that relates occurrences to an absolute distance (e.g., nucleotide number), thereby producing a distance vs. frequency of occurrence relationship. Frequencies of occurrences observed in a test nucleic acid can be compared to the distance standard to determine absolute distances. Two or more nucleic acids of unknown structure can also be compared directly to determine differences in frequencies of occurrences that can be interpreted as differences in distances (relative distances). This is useful to detect differences in two or more nucleic acids presumed to be highly similar. For example, genomes of unknown structure from a normal cell and a tumor cell from common genetic origins (e.g., from the same individual) may be compared directly to determine differences in distances. Differences in distances in this context may be relevant to understanding contributing genetic factors to development of the cancer. For example, difference in distances may be the result of a genetic aberration, such as an insertion or deletion, in a cancer related gene. Other applications will be apparent to the skilled artisan.

A variety of methods known in the art can be used for preparing nucleic acid fragments from nucleic acids. As used herein, “fragmenting” refers to the preparation of nucleic acids of a smaller size than a starting (template) larger nucleic acid. Fragmentation may occur as part of or following a harvest method. Fragmenting can occur by any number of means and the invention is not to be limited in this regard. For example, fragmenting can occur enzymatically, mechanically (e.g., via shearing), or chemically. Examples of enzymatic fragmenting include digestion with one or more nucleases whether sequence specific (e.g., restriction endonuclease) or sequence non-specific (e.g., micrococcal nuclease, mung bean nuclease, DNase I). An example includes DNase I. One of ordinary skill will appreciate that the conditions for enzymatic digestion will vary depending on the degree of fragmentation and the length of fragments ultimately desired. For example, the concentration of enzyme and/or any required co-factors, the temperature of the digestion reaction, and the length of the digestion reaction can be varied singly or in combination to achieve the desired degree of fragmentation. As an example, digestion with DNase I at 25-37° C. for 1-2 minutes may be used to generate a population of genomic target nucleic acids ranging in size from about 5-1000 bps. Determination of other conditions is within the skill of the ordinary artisan.

Further examples of enzymatic fragmenting include performing linear extension polymerase (e.g., DNA polymerase, RNA polymerase) reactions on a nucleic acid. Such reactions can be performed using random primers (e.g., using random hexamers). Alternatively, such reactions can be performed using specific primers. For example, template nucleic acids may be first digested, for example with a restriction enzyme, and linkers/adapters can be ligated to the digested templates to produced linker/adapter ligated nucleic acids. Specific primers complementary to the linker/adapters can then be used to prime a linear extension reaction. In other examples, random lengths can be produced by controlling the elongation time (e.g., processivity of the enzyme). Polymerase has a tendency to “fall off” the template at random positions on the template nucleic acid thereby producing random fragment lengths. It is understood the tendency to fall off (and thereby the fragment length) can be manipulated by adjusting various reaction parameters such as salt concentration, temperature, nucleotide concentrations, etc. In some cases, extensions can be controlled to produce random fragments by adding dideoxynucleotides (ddNTP) to the linear extension reaction. The fragment lengths can be modulated by the dideoxynucleotide concentration.

Examples of mechanical fragmenting include shearing as can occur using sonication, nebulization, HPLC, and use of a French press or a Hydro Shear device (GeneMachines, San Carlos, Calif.), and the like. Sonication may be performed by exposing nucleic acids to a sonicator as described by Bankier and Barrell 1987 Meth. Enzymol. 155, 51-93. Sonicators are commercially available from for example Misonix Inc. (Farmingdale, N.Y.). Nebulization refers to the use of hydrodynamic shearing forces to fragment nucleic acids. This can be accomplished for example by flowing a nucleic acid through a constriction in a flow pathway such as a tube or microfluidic channel. The size of the constriction and the volume of fluid through the constriction can be modified to achieve the desired degree of fragmentation. Nebulizers are commercially available from GeneMachines (San Carlos, Calif.). Reference also be made to U.S. Pat. Nos. 5,506,100 and 5,610,010.

Examples of chemical fragmenting include incubation with chemicals such as piperidine, piperidine with hydrazine or dimethyl sulfate, hydrogen peroxide, phenanthroline, and the like. Some methods of the invention may combine these techniques. For example, genomic DNA may be sonicated and digested with one or more restriction endonucleases to generate fragments of a desired size range.

The target nucleic acids may be isolated and/or purified following fragmentation using any method of choice. For example, the target nucleic acids may be cleaned by ethanol precipitation, agarose gel purification, RNase treatment to remove RNA from the sample (or DNase treatment to remove DNA), mild centrifugation to pellet nucleic acid fragments leaving nucleotides and oligonucleotides (up to for example 50 bp in solution), column chromatography, and the like, including some combination thereof. Purification may be performed using commercially available clean up kits including but not limited to QiaPrep (Qiagen, Valencia, Calif.).

Target nucleic acids of the desired length ranges can be isolated from nucleic acids that are longer or shorter. This can be accomplished using techniques known in the art including but not limited to agarose gel purification, size exclusion chromatography, SPRI (Agencourt Bioscience, Beverly Mass.), column separation, and the like. Those of ordinary skill will appreciate that the target nucleic acids can be both purified and size selected using the same technique (e.g., agarose gel purification).

Nucleic acid fragments produced by the methods disclosed herein comprise only one predefined region having a sequence complementary to a predefined location of the nucleic acid. A predefined region is a region in a nucleic acid fragment from which the distance to a second region in the nucleic acid fragment (a test region) is measured. Nucleic acid fragments are processed such that each fragment has a predefined region. The predefined region in a nucleic acid fragment corresponds to a predefined location in the nucleic acid. The predefined location is a position in the nucleic acid where a predefined sequence (e.g., a restriction site) occurs. Predefined sequences (and therefore predefined locations and regions) may occur in a nucleic acid at a predefined frequency. For example, a predefined sequence that is a hexamer sequence will occur at a frequency of ¼^{̂6} or 1 in 4096 bases.

Nucleic acid fragments can be prepared such that each fragment has a predefined region by any one of a number of methods. In one embodiment, nucleic acids are digested with a restriction enzyme prior to fragmenting. In one embodiment, nucleic acids are digested with a restriction enzyme after fragmenting. Digestion with a restriction enzyme results in fragments having a predefined region at a fragment end. Thus, predefined sites can be any one of a number restriction sites known in the art that are defined by the specificity of a restriction enzyme. Exemplary sites include those recognized by the following Restriction Enzymes: AatII, Acc65I, AccI, AciI, AclI, AcuI, AfeI, AflII, AflIII, AgeI, AhdI, AleI, AluI, AlwI, AlwNI, ApaI, ApaLI, ApeKI, ApoI, AscI, AseI, AsiSI, AvaI, AvaII, AvrII, BaeGI, BaeI, BamHI, BanI, BanII, BbsI, BbvCI, BbvI, BccI, BceAI, BcgI, BciVI, BclI, BfaI, BfuAI, BfuCI, BglI, BglII, BlpI, Bme1580I, BmgBI, BmrI, BmtI, BpmI, Bpu10I, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssSI, BstAPI, BstBI, BstEII, BstNI, BstUI, BstXI, BstYI, BstZ17I, Bsu36I, BtgI, BtgZI, BtsCI, BtsI, Cac8I, ClaI, CspCI, CviAII, CviKI-1, CviQI, DdeI, DpnI, DpnII, DraI, DraIII, DrdI, EaeI, EagI, EarI, EciI, EcoNI, EcoO109I, EcoP15I, EcoRI, EcoRV, FatI, FauI, Fnu4HI, FokI, FseI, FspI, HaeII, HaeIII, HgaI, HhaI, HincII, HindIII, Hinfl, HinP1I, HpaI, HpaII, HphI, Hpy166II, Hpy188I, Hpy188III, Hpy99I, HpyAV, HpyCH4III, HpyCH4IV, HpyCH4V, KasI, KpnI, MboI, MboII, MfeI, MluI, MlyI, MmeI, MnlI, MscI, MseI, MslI, MspA1I, MspI, MwoI, NaeI, NarI, Ncil, NcoI, NdeI, NgoMIV, NheI, NheI-HF™, NlaIII, NlaIV, NmeAIII, NotI, NruI, NsiI, NspI, PacI, PaeR7I, PciI, PflFI, PflMI, Phol, PleI, PmeI, PmlI, PpuMI, PshAI, PsiI, PspGI, PspOMI, PspXI, PstI, PvuI, PvuII, PvuII-HF™, RsaI, RsrII, SacI, SacII, SalI, SalI-HF™, SapI, Sau3AI, Sau96I, SbfI, Scal, ScaI-HF™, ScrFI, SexAI, SfaNI, SfcI, SfiI, SfoI, SgrAI, SmaI, SmlI, SnaBI, SpeI, SphI, SphI-HF™, SspI, StuI, StyD4I, StyI, SwaI, TaqαI, TfiI, TliI, TseI, Tsp451, Tsp509I, TspMI, TspRI, Tth111I, XbaI, XcmI, XhoI, XmaI, XmnI, and ZraI. Other suitable restriction sites and corresponding restriction enzymes will be known to the skilled artisan.

In some embodiments, predefined locations are primer recognition sites and a nucleic acid (or nucleic acid fragments) can be processed in a linear extension polymerase reaction using such primers to produce fragments having predefined regions at one end. Primers can be designed having any desired sequence provided the primer is capable of initiating an extension reaction. Primer length can be adjusted to alter the frequency of occurrence of predefined locations in a nucleic acid. For example, a primer that is a hexamer sequence will occur at a frequency of 1 in 4096 nucleotides. Whereas a primer that is a octamer sequence will occur at a frequency of 1 in 65536 nucleotides. In some embodiments, the primer length is up to 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides in length. In some embodiments, the following primers are used *(SEQ ID NO: 4) GGGCTGGAGAGATGGC, (SEQ ID NO: 5) GAGATATTAATTGGCT, (SEQ ID NO: 6) GCCAATGGCTGGGCAG, (SEQ ID NO: 7) GAAATGCAAATCAAAA, (SEQ ID NO: 8) TGGCGAGGATGTGGAG, (SEQ ID NO: 9) GCTCCACTATGTTCAT, (SEQ ID NO: 10) CAGTATTGTTTTATTA, (SEQ ID NO: 11) GAAACTTAGTCTCCTG, (SEQ ID NO: 12) CATTATATGGATGATA, (SEQ ID NO: 13) AGTTCGAGGCCAGCCT, (SEQ ID NO: 14) GAGTTCGAGGCCAGCC, (SEQ ID NO: 15) CCAGCACTCGGGAGGC or CTGTCC.

The invention, in some aspects, is based on the development of methods for producing pools of nucleic acid fragments that have a distribution of sizes, and the distance between any position (e.g., a test region) within a nucleic acid fragment and a predefined region of the fragment is inversely related to the frequency of occurrence of the position (e.g., the test region) in the distribution. The distribution of sizes of nucleic acid fragments can be referenced to infer distances between locations (e.g., predefined locations, test locations) in the nucleic acid. A variety of methods can be used to measure the frequency of occurrence of test regions in pools of nucleic acid fragments. In some embodiments, the methods involve contacting the nucleic acid fragments with one or more polynucleotides under conditions appropriate for hybridization of nucleic acid fragments with the polynucleotides, wherein each query probe comprises one or more polynucleotides having a sequence complementary to a test location of the nucleic acid, and assessing the extent of hybridization of the nucleic acid fragments with the one or more polynucleotides, wherein the extent of hybridization is indicative of the frequency of occurrence of nucleic acid fragments in the pool of nucleic acid fragments having a sequence complementary to the test location of the nucleic acid. This frequency of occurrence can then be related to the distance between the test location and the predefined location. The extent of hybridization of polynucleotides with nucleic acid fragments can be assessed by any approach known in the art for evaluating hybridization events. In some embodiments, polynucleotides are fixed to a solid support and arranged in an array format to produce a ruler array.

Ruler array technology provides a high throughput method to measure genomic distances across an entire genome with a single experiment and can be used to examine rearrangement of, for example, tandem repetitive elements. In a ruler array experiment points of illumination, or fluorescence, are placed along the genome, and the intensity of this illumination, or fluorescence, signal is measured at tiled genomic positions by a DNA microarray. Thus, each probe on the array measures the distance between that probe's sequence and the closest points of illumination that have been selected on the genome. A ruler array can be used to approximate absolute distances in a single genome, and can also be used with two-color DNA microarrays to detect variations between two genomes. In this second application, one can measure genomic changes between a control strain and a strain that has been subject to environmental stress. In another example, genomic changes between a control genome, also referred to as a reference genome, and a test genome can be compared.

In one embodiment ruler arrays measure the genomic distance between two defined sequences one of which is encoded in the query probe and one is a defined site in the genome of interest. In one embodiment, ruler arrays detect changes in distance between unique sequence elements at a resolution of up to about 1 kb, about 1 to about 10 kb, about 10 kb to about 100 kb, or more than 100 kb. In one embodiment, distances between unique sequence elements at are detected at a resolution of between about 3 kb and about 5 kb.

As used herein, ruler arrays are arrays of query probes used to determine the frequency of occurrence of test regions in nucleic acid fragments. Ruler arrays can be used to measure the distance between specific unique sequences (locations) in a nucleic acid. For example, in a single experiment, a ruler array can measure genomic distances between many pairs of sequence specified unique locations. Ruler arrays have wide application to the study of genome evolution. Ruler arrays have direct medical importance, and facilitate the study of how pathogenic organisms evolve their genome to better adapt to their host environment and avoid host defenses. In one embodiment, ruler arrays examine genomic changes associated with the development of multicellular organisms, and can provide quantitative genetic insight at the level of cell growth or differentiation. In one embodiment ruler arrays examine genomic changes associated with genetic diseases, such as cancer.

As used herein, a query probe comprises one or more identical polynucleotides that identifies, by sequence complementarity, a known region in a reference nucleic acid. A query probe sequence is often a unique genome sequence that defines a test location. In one embodiment a query probe comprises one or more common polynucleotides that are each fixed at one end to a solid support. In one embodiment, query probes are arranged in an array format, wherein multiple distinct query probes are arrayed on a solid support, wherein each distinct query probe is a located at an addressable location, and wherein the sequence information associated with each distinct query probe is stored in a computer readable format. In one embodiment, a set of query probes comprises at least three different polynucleotides, each of whose sequence identifies a known region in a reference nucleic acid. In some embodiments, a ruler array comprises query probes, also referred to as ruler probes, wherein each query probe comprises one or more polynucleotides fixed to a solid support. A query or ruler probe may include spacer sequences, which are, for example, located at least one end of a query probe and useful to attach a query probe to a solid support, such as a microarray. The term microarray includes a variety of formats, such as a flat surface, spherical or ellipsoid support or any other appropriate support for at least one query probe. For example, many spheres each of which bears at least one query probe, can be used. In some embodiments, a ruler array comprises up to 10, up to 100, up to 1000, up to 10000, up to 100000, or more query probes. However, ruler arrays are not so limited.

A query probe is useful to measure genomic distance of randomly sheared DNA or randomly fragmented DNA. This is the case because after DNA is sheared or fragmented, the number of DNA molecules that contain two unique sequences will be inversely related to the distance between the two sequences (e.g., test locations and predefined locations). When the sequences are close together, it is likely that fragmenting will not disassociate them and there will be a large number of DNA molecules with both sequences. When the two sequences are far apart, fragmenting is likely to disassociate them, and there will be a correspondingly small number of DNA molecules. In one embodiment, fragmentation of DNA is accomplished by sonication.

In one embodiment, ruler arrays use nucleic acid (e.g., genomic DNA) features referred to as predefined sites, such as the position of restriction sites, as one member of a pair of specific sequence that is used to measure distances. As used herein, distance is the number of bases between a pair of sequence specific sites in a nucleic acid, such as a genomic DNA.

To provide absolute distances, control (or reference) query probes can be used to provide a calibration source when given DNA of known and constant sequence. A control query probe can be located in a portion of a genome where distance changes would be deleterious, such as in the coding regions of selected genes.

To provide relative distances between two DNA samples, the samples can be labeled with different fluorescent labels and hybridized to the same array, such as a microarray. The ratios (or relative fluorescence) at each ruler probe will give the relative change in distance between the two samples.

Ruler array methods can be implemented using any commercial microarray. Available commercial manufacturers include Agilent, Nimblegen, and Affymetrix. In some embodiments, disclosed herein Agilent's 244k S. cerevisiae design, part number G4491A is used. Ruler array methods can be implemented using any tiling array design, regardless of whether it was intended for ChIP-Chip, CGH, or tiling expression experiments. As disclosed herein, the criteria used to design or pick probes (polynucleotides sequences) for use in the ruler array methods are similar to those used to pick probes for other arrays (eg CGH or ChIP-Chip). For example, probe spacing should be roughly uniform across the nucleic acid in which length is being measured, and probe sequences should be unique. In some cases, short matches to unintended locations have little effect on results while long matches may result in that a probe's intensity being the sum of the intended and unintended intensities (the probe queries multiple genomic locations simultaneously). Typically, probes on the array should have similar melting temperature and should not form secondary structures that might preclude binding to the labeled sample.

As disclosed herein, array based methods for assessing the occurrence of test regions in pools of nucleic acid fragments involve labeling of fragments. Fragments can be labeled by any appropriate methods known in the art. For example, array manufacturer's, such as Affymetri, provide labeling instructions that are appropriate in many cases, as will be apparent to the skilled artisan. Labeling methods may be primer directed or restriction site directed. For example, adapters that are ligated to restriction enzyme digested fragments can be labeled directly (e.g., conjugated to a detectable label, including a detectably labeled nucleotide) to produce fragments having a single label. In other embodiments, fragments are uniformly labeled. For example, during any of the primer extension reactions disclosed herein, detectably labeled nucleotides can be included in the reaction mixture to incorporate labeled nucleotides directly in the fragments. Primer extension labeling technique use one or more primers directed against a nucleic acid (or nucleic acid fragment) or adapter sequence and incorporate detectably labeled nucleotides in a nucleic acid fragment during elongation. In some embodiments, the primer itself may be labeled and detectably labeled nucleotides may or may not be incorporated into the labeled nucleic acid fragment.

Another labeling strategy uses a nicking enzyme, such as BsmI and a polymerase that can initiate from the nick and that has a strong strand displacement ability, such as Bst, that can incorporate labeled nucleotide(s) during the polymerase reaction. Other nicking enzymes include Nb.BbvCI, Nb.BsmI, Nb.BsrDI, Nb.BtsI, Nt.AlwI, Nt.BbvCI, Nt.BspQI, Nt.BstNBI, Nt.CviPII. Still others will be apparent to the skilled artisan.

Labeled nucleotides can be labeled with fluorescent dyes including but not limited to fluorescein, pyrene, 7-methoxycoumarin, Cascade Blue™, Alexa Flur 350, Alexa Flur 430, Alexa Flur 488, Alexa Flur 532, Alexa Flur 546, Alexa Flur 568, Alexa Flur 594, Alexa Flur 633, Alexa Flur 647, Alexa Flur 660, Alexa Flur 680, AMCA-X, dialkylaminocoumarin, Pacific Blue, Marina Blue, BODIPY 493/503, BODIPY FI-X, DTAF, Oregon Green 500, Dansyl-X, 6-FAM, Oregon Green 488, Oregon Green 514, Rhodamine Green-X, Rhodol Green, Calcein, Eosin, ethidium bromide, NBD, TET, 2′, 4′, 5′, 7′ tetrabromosulfonefluorescien, BODIPY-R6G, BODIPY-FI BR2, BODIPY 530/550, HEX, BODIPY 558/568, BODIPY-TMR-X., PyMPO, BODIPY 564/570, TAMRA, BODIPY 576/589, Cy3, Rhodamine Red-x, BODIPY 581/591, carboxyXrhodamine, Texas Red-X, BODIPY-TR-X., Cy5, SpectrumAqua, SpectrumGreen #1, SpectrumGreen #2, SpectrumOrange, SpectrumRed, or naphthofluorescein. Other appropriate dyes are known in the art.

In some cases, it may be desirable to amplify nucleic acid fragments (e.g., during labeling) by PCR using a thermostable polymerase, which is an enzyme that synthesizes nucleic acids and is relatively intolerant to temperature changes, including repeated temperature changes, ranging from room temperature to 94° C. Thermostable polymerases are well known in the art and include recombinant and non-recombinant polymerases as well as polymerases with and without 3′-5′ exo-nuclease activity. Non-limiting examples of thermostable polymerases include Hot Start polymerase, Pfu DNA polymerase, Tbr DNA polymerases, Tfl DNA polymerases, Tgo DNA polymerases, Tth DNA polymerases, Taq polymerases, Vent polymerase, Platinum HiFi Taq, Stearothermophilus polymerase I, and the like. The PCR reaction may include labeled nucleotides in combination with unlabeled nucleotides (dNTPs). In some embodiments, dNTPs are selected from the group consisting of naturally occurring dNTPs (dCTP, dATP, dGTP, dTTP, and dUTP). In some embodiments, the dNTPs are dCTP, dATP, dGTP and dTTP. In some embodiments, dUTP is added to that mixture. In other embodiments, one or more non-naturally occurring dNTP are used instead of or in addition to naturally occurring dNTP. These include an analog of a dNTP, a modified dNTP, a dNTP having a universal base, and the like.

Other methods for measuring the frequency of occurrence of test regions in pools of nucleic acid fragments include sequencing. The methods use short sequencing reads, for example, sequences produced by a Solexa machine or similar system known in the art. The incidence of unique sequences that appear in the short sequencing reads are used to establish the frequency of occurrence of test regions. Sequencing reactions may be primers complementary to internal sequences of nucleic acid fragments. In some embodiments, query probe sequences can be used to prime sequencing reactions. In other embodiments, adapters are ligated on the end opposite of the predefined sequence of nucleic acid fragments and primers complementary to the ligated adapters are used to prime the sequencing reactions, thereby sequencing the ends of fragments farthest from the predetermined site.

After sequencing the ends of the nucleic acid fragments, the resulting sequencing reads may be mapped back to a nucleic acid reference sequence, and “virtual array intensities” can be generated by extending each fragment from its read back to the predefined location in the nucleic acid. The virtual array intensity at any point is the number of extended sequencing reads (number of fragments) that cross that point. These virtual intensities can be processed in the same manner as actual array intensities since the intensities measured on the microarray increase linearly with the number of fragments that include the microarray probe, in the same way that the virtual intensity at some point increases linearly with the number of fragments that included that point

### Distance Analysis

One embodiment is a method for detecting a difference between distance between two sequence specified locations in a test nucleic acid and distance between the same two sequence specified locations in a reference nucleic acid. In this context, “distance” refers to the number of bases between two sequence-specified locations in a nucleic acid. One of the two sequences is specified by a site (referred to as a “defined site” or “label site”) at which a detectable label is introduced (e.g., restriction enzyme recognition site). The second of the two sequences is specified by a polynucleotide (referred to as a “query probe”) with a sequence that identifies a known region in the reference nucleic acid. Distances in the reference nucleic acid are known for a particular set of query probes and a particular defined site. In contrast, distances in the test nucleic acid are unknown. The method makes use of a reference hybridization pattern. This reference hybridization pattern is used to establish a relationship between the extent of hybridization (EOH) at each query probe and the distance from each query probe to defined sites.

In one embodiment, distance is determined (distance analysis is carried out) as follows: A collection of labeled test nucleic acid fragments is hybridized to a set of query probes (e.g., a genome array). The extent of hybridization (EOH) of labeled test nucleic acid fragments at each query probe is measured and the EOH of labeled test nucleic acid fragments at each query probe is associated with the corresponding region identified by the query probe in the reference nucleic acid and the corresponding location of defined sites in the reference nucleic acid. The presentation of these data produces a test hybridization pattern, which is evaluated against (with respect to) a reference hybridization pattern and associated distances. This evaluation makes it possible to determine unknown distances in the test hybridization pattern. A difference between distance in a test nucleic acid and distance in a reference nucleic acid is detected. In one embodiment, distance analysis is repeated, as needed, to detect multiple differences in distance.

### Ratio Analysis:

One embodiment is a method for detecting a difference between a test nucleic acid and a reference nucleic acid by direct comparison of hybridization patterns and is carried out as follows: A collection of labeled test nucleic acid fragments is hybridized to a set of query probes (e.g., a genome array). The extent of hybridization (EOH) of labeled test nucleic acid fragments at each query probe is measured and the EOH of labeled test nucleic acid fragments at each query probe is associated with the corresponding region identified by the query probe in the reference nucleic acid. The presentation of these data produces test hybridization pattern. A ratio hybridization pattern is produced that reflects the relative EOH of labeled test nucleic acid fragments to EOH of labeled reference nucleic acid fragments at each query probe. A Significant local maxima or a significant local minimum in the ratio hybridization pattern is detected and reflects a location of difference between the test and reference nucleic acids. Significant local maxima or minima are considered to be maxima or minima that respectively define the peak or valley of a broadly shaped curve, which represents a set of data points that deviate significantly from the value reflecting equivalence between test and reference nucleic acid patterns in a common direction relative to the value reflecting equivalence between test and reference nucleic acid patterns. Thus, typically significant local maxima or minima are closely surrounded by one or more ratio data points that are respectively greater than or less than the value reflecting equivalence between test and reference nucleic acid patterns. In one embodiment, ratio analysis is repeated, as needed, to detect multiple differences.

In one embodiment, a ratio analysis detects difference between a test nucleic acid and a reference nucleic acid, and a subsequent distance analysis is performed to determine distances in the test nucleic acid at each difference detected in the ratio analysis.

In one embodiment, the nucleic acid is DNA from a genome of interest. In one embodiment, the location of defined sites in the reference nucleic acid is known and available in a computer readable format.

The hybridization pattern is determined by associating measurements of the extent of hybridization (EOH) of labeled nucleic acid fragments at each query probe with the corresponding region identified by each query probe in the reference nucleic acid and the corresponding location of defined sites in the reference nucleic acid. A test hybridization pattern is generated using EOH measurements with labeled test nucleic acid fragments. A reference hybridization pattern is generated using EOH measurements with labeled reference nucleic acid fragments. A reference hybridization pattern need not be determined or established simultaneously or concurrent with the generation of a test hybridization, but may already be known and accessible for analysis (a pre-existing reference). In one embodiment a reference hybridization pattern that describes the relationship between EOH measurements and distance (also, referred to as expected intensity vs distance relation) is determined by averaging over all known labeling sites in a test nucleic acid dataset. Hybridization patterns are at least dependent on the method used to produce the labeled nucleic acid fragments, the location of query probes, and the location of the defined site(s).

The distance from a query probe to a defined site is applicable to subsequent analysis when the query probe is within the resolution limit of the defined site. The resolution limit is the maximum distance that label incorporated at a defined site into nucleic acid fragments will be detectable by hybridization of the corresponding labeled nucleic acid fragments with a query probe. The resolution limit is at least dependent on fragmentation methods, labeling methods, hybridization conditions, query probe design, and characteristics of the label detection system. One aspect that influences resolution is the distance in bases between consecutive query probes. In one embodiment, the distance between consecutive query probes is less than about 100 bases. In one embodiment the distance between consecutive query probes is between about 100 and 1000 bases. In one embodiment the distance between consecutive query probes is between about 1000 and 100,000 bases. In one embodiment the distance is between consecutive query probes is greater than 100,000 bases.

Several methods are provided for obtaining a collection of labeled test nucleic acid fragments. In one example, fragmentation of nucleic acids is accomplished by sonication; and labeling of the nucleic acid fragments is accomplished by restriction enzyme digestion, linker ligation, and ligation-mediated linear PCR using fluorophore conjugated nucleotides. The distribution of lengths in the collection of labeled test nucleic acid fragments is known or can be determined using known methods and is essentially equivalent to the distribution of lengths in the collection of labeled reference nucleic acids.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The PolymeRase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The present invention is illustrated by the following examples, which are not intended to be limiting in any way.

### EXAMPLES

### Example 1

### Materials and Methods

### Nucleic Acid Labeling

### Sonicate Genomic DNA

1. Add water and 3M NaOAC to make 700 ul of 0.3M NaOAc and sonicate 1×15 seconds at power level 1.
2. Ethanol precipitate, spin, wash with 80% ethanol, spin, air dry for 5 minutes and resuspend WELL in water.
3. Run sonicated DNA on a gel to ensure that DNA fragments have a mean length of approximately 3-5 KB.

### Option A: Label at Restriction Sites

1. Digest DNA with selected enzyme(s) for 2 hrs at 37 degrees with 2-fold excess of enzyme. Ensure enzymes have a compatible buffer.
2. Add 10×CIP buffer to 0.5× final concentration and 2-fold excess CIP. Incubate 1 hr at 37 degrees.
3. Phenol extract with equal volume phenol.
4. Phenol/chloroform/isoamyl alcohol extract with equal volume of phenol/chloroform/isoamyl alcohol.
5. Ethanol precipitate with 3 volumes ethanol. Spin 15 K for 15′, wash with 80% ethanol, spin as above and air dry for 5 minutes.
6. Resuspend in water and OD 260/280.
7. Ligate overnight at 14 degrees with annealed oligos.
8. Qiagen column purify ligation mix and elute column with 50-100 ul water.
9. Using a primer that is compatible with annealed oligos, linearly amplify and label with low T mix and cy 5 or 3.

| | | |
|---|---|---|
| 94 degrees | 2′ | |
| 94 degrees | 1′ | |
| 57 degrees | 30″ | 20X yeast, 25X mouse |
| 72 degrees | 3′30″ | |
| 72 degrees | 5′ | |

10. Qiagen column purify, elute in 50 ul and OD on the nanodrop for DNA concentration and label concentration.

### Option B: Label at Oligo Defined Sites

1. Linearly label with low T mix and cy 5 or 3. Molar ratio of dTTP to Cy-dTTP should be 3:1. Primer mix is a defined by oligos that mark desired illumination points in the genome.

| | | |
|---|---|---|
| 94 degrees | 2′ | |
| 94 degrees | 1′ | |
| 50 degrees | 45″ | 30X |
| 72 degrees | 3′30″ | |
| 72 degrees | 5′ | |

2. Qiagen column purify, elute in 50 ul and OD on the nanodrop for DNA concentration and label concentration.

### Ligation Protocol

1. Cut DNA (25 ug) with enzyme(s). 3 hours at 37 C using 2× or 3× excess of enzyme.
2. Add to that mix 10×CIP buffer and appropriate amount of CIP. Incubate 1 hr at 37 C.
3. Phenol chloroform extract and precipitate
4. Wash and OD. End up with about 80% of the input DNA.
5. Ligate on preannealed oligo(s) (anneal by mixing comparable amounts at pH 8. Heat for 5 min to 95 C, put in 70 C heat block and remove to bench letting cool to room temp. When gets to room temp, keep in block and store at 4 C overnight. Aliquot and freeze overnight). Try to use 2× concentration of oligo compared to concentration of ends. Incubates at 14 C overnight using T4 DNA ligase.
6. Run on Qiagen column to get rid of loose oligo and enzymes. This usually filters out anything below ˜50 bp.
7. Recut with BamHI if doing that control experiment
8. Sonication step
   a. First yeast experiment was power 4 2×15 s
   b. Second yeast experiment was power 4 1×15 s
   c. First mouse experiment was power 1 1×10 s
9. PCR
   Yeast: PCR with 2 ul of 5 mM G, A, C, 2 mM T and 2 ul of Cy labeled dTTP (need to look up the concentration of this).
      94 C 2 min
      94 C 1 min
      57 C 30 s
      72 C 2 min
      go back to #2 20×
      72 C 5 min
      Clean up on Qiagen column
      OD with nanodrop—gives OD and amount of Cy dye incorporated. Yeast arrays have used 20 pMoles dye per channel, usually between 2 and 5 ug of DNA
10 Mouse: Same as yeast, except 25 cycles instead of 20
   94 C 2 min
   94 C 1 min
   57 C 30 s
   72 C 3:30 s
   go back to #2 25×
   72 C 5 min
   Clean up on Qiagen
   OD and nanodrop. Used 20 pMoles dye per channel, 2 to 7 ug of DNA per channel
11. Hybridize over weekend (>42 h) at 65 C
12. Wash and scan

### Multi Oligo Protocol

1. Sonicated 3×10 s at power 1
2. Took 4 ug of DNA
3. PCRed using 8 primers
   94 C 2 min
   94 C 1 min
   50 C 45 s
   72 C 3:30 s
   go to 2 25×
   72 C 5 min
4. Cleaned up on Qiagen
5. Nanodrop and OD. Used 20 pMoles label on array

### Digestion-Ligation-Label Protocol

1. Eco R1 digest DNA for 2 hrs at 37 degrees with 2-fold excess of enzyme.
2. Add 10×CIP buffer to 0.5× final concentration and 2-fold excess CIP. Incubate 1 hr at 37 degrees.
3. Phenol extract with equal volume phenol.
4. Phenol/chloroform/isoamyl alcohol extract with equal volume of phenol/chloroform/isoamyl alcohol.
5. Ethanol precipitate with 3 volumes ethanol. Spin 15 K for 15′, wash with 80% ethanol, spin as above and air dry for 5 minutes.
6. Resuspend in water and OD 260/280.
7. Ligate overnight at 14 degrees with annealed oligos.
8. Qiagen column purify ligation mix and elute column with 50-100 ul water.
9. Add water and 3M NaOAC to make 700 ul of 0.3M NaOAc and sonicate 1×15 seconds at power level 4.
10. Ethanol precipitate, spin, wash with 80% ethanol, spin, air dry for 5 minutes and resuspend WELL in water.
11. PCR with low T mix and cy 5 or 3.

| | | |
|---|---|---|
| 94 degrees | 2′ | |
| 94 degrees | 1′ | |
| 57 degrees | 20″ | 20X |
| 72 degrees | 3′30″ | |
| 72 degrees | 5′ | |

12. Qiagen column purify, elute in 50 ul and OD on the nanodrop for DNA concentration and label concentration.

Primers for Digest/Ligate Protocol:
EcoRI adapter:

| | |
|---|---|
| AATTGGAGGAGGGAAGGGGG | (SEQ ID NO: 1) |
| NcoI adapter: | |
| CATGGGAGGAGGGAAGGGGG | (SEQ ID NO: 2) |
| primer for EcoRI and NcoI | |
| CCCCCTTCCCTCCTCC | (SEQ ID NO: 3) |

For the primers + genomic DNA + PCR protocol, we
used a number of primers at once:

| | |
|---|---|
| GGGCTGGAGAGATGGC | (SEQ ID NO: 4) |
| GAGATATTAATTGGCT | (SEQ ID NO: 5) |
| GCCAATGGCTGGGCAG | (SEQ ID NO: 6) |
| GAAATGCAAATCAAAA | (SEQ ID NO: 7) |
| TGGCGAGGATGTGGAG | (SEQ ID NO: 8) |
| GCTCCACTATGTTCAT | (SEQ ID NO: 9) |
| CAGTATTGTTTTATTA | (SEQ ID NO: 10) |
| GAAACTTAGTCTCCTG | (SEQ ID NO: 11) |
| CATTATATGGATGATA | (SEQ ID NO: 12) |
| AGTTCGAGGCCAGCCT | (SEQ ID NO: 13) |
| GAGTTCGAGGCCAGCC | (SEQ ID NO: 14) |
| CCAGCACTCGGGAGGC | (SEQ ID NO: 15) |

The foregoing primers were used to analyze mouse genomic DNA.

For the primers + genomic DNA + PCR protocol, we
used a number of short primers at once:
*CAGAGG
*CTGGGA

The foregoing short primers were used to analyze genomic DNA (e.g., mouse genomic DNA).

### Bead-Based Labeling Protocol

We used a biotinylated adapter molecule (same as above but with GGGG-biotin added to the 3′ end).

We modified the protocol:
digest
ligate
sonicate
mix with streptavidin beads
wash off unbound material
PCR with primer (this is done with the template still attached of the beads)

### Nick-Displacement Protocol

Use a nicking enzyme (BsmI in our case) and a polymerase that can initiate from the nick and has a strong strand displacement ability (Bst in our case). This allows for an isothermic reaction, in which there is a continual nicking and copying. The labeling sites in this protocol are also defined by the nicking enzyme. In comparison with other protocols there is (1) no ligation, which in some cases can be inefficient and (2) no cycling, which in some cases can reduce the time to incorporate the labeled nucleotides.

### Labeling Protocols

In the Digest-Ligate-Label-Hybridize protocol we first digest the genomic DNA with one or several restriction enzymes that leave sticky ends. We then add adapter oligos that contain (1) a 5′ sequence complementary to the sticky end and (2) an arbitrary 3′ end chosen for our convenience. We use a partially double-stranded oligo pair such that part (1) is single stranded and part (2) is double stranded. After the ligation, the longer adapter molecule is firmly attached to the genomic DNA while the shorter primer oligo may disassociate. We then add more of the shorter oligo to prime a PCR extension to incorporate labeled nucleotides. This primer will hybridize to, and thus prime, the adapter molecule ligated onto the restriction enzyme sites as well as any genomic loci to which it is complementary. Typically, we analyze the labeling of genomic DNA on one side of the restriction site. However, the reaction will label in both directions on opposite strands.

The primer extension labeling technique uses one or more oligos directed against genomic DNA (without the digestion and ligation steps). By using a relatively long oligo (e.g., SEQ ID NO: 16 GATCCGAATTCTGTCC), the amplication targets specific genomic loci. While this may provide data over a relatively small fraction of a genome, it makes insertions or deletions of the labeled site extremely obvious. This technique would be useful if the oligo or oligos label sites contained in transposable elements or other sequences suspected of changing between two genomic samples.

Using short sequences to prime a PCR reaction that incorporates labeled nucleotides is similar to using long oligos, except that more genomic locations will be labeled when short sequences are used. Using hexamers (e.g., CTGTCC), for example, should label roughly as many sites as a restriction enzyme that recognizes a six nucleotide sequence, but the hexamer offers more flexibility. In particular, we might choose a hexamer whose genomic locations are more uniformly distributed through the genome than any available restriction site, thus providing data about a larger fraction of that genome.

A variation on the Digest/Ligate protocol uses an oligo into which dye has been incorporated prior to the ligation (addition of the adapter). Pre-labeling the oligo removes the need for the PCR step and has the added advantage of incorporating the same amount of dye at each restriction site.

Another labeling strategy uses a nicking enzyme, such as BsmI and a polymerase that can initiate from the nick and that has a strong strand displacement ability, such as Bst, that can incorporate labeled nucleotide(s) during the polymerase reaction. Other nicking enzymes include Nb.BbvCI, Nb.BsmI, Nb.BsrDI, Nb.BtsI, Nt.AlwI, Nt.BbvCI, Nt.BspQI, Nt.BstNBI, Nt.CviPII. Still others will be apparent to the skilled artisan.

### Other Protocol Variations

The ruler array technique requires a population of nucleic acid fragments with some distribution of lengths and involves:
digesting an input DNA sample
ligating an adapter to the digested material
sonicating the sample
extending from a primer complementary to the adapter to generate labeled fragments
hybridizing to an array

### Biotin Purification of Ligated Material

We used a biotinylated adapter molecule to separate the successfully ligated fragments from the remainder. Since ligation has a low efficiency (perhaps 10%), the majority of the material in the sonication, extension, and hybridization might have been unligated, unlabeled template. The purification allows us to include only the labeled extension product in the hybridization.

| | |
|---|---|
| | (SEQ ID NO: 16) |
| | genomic dna |
| Starting | AGTGGGACGTGGACAGAATTCGGATC |
| | (SEQ ID NO: 17) |
| | TCACCCTGCACCTGTCTTAAGCCTAG |
| | genomic dna |
| | (SEQ ID NO: 18) |
| | genomic dna |
| Digest | AGTGGGACGTGGACAG |
| | (SEQ ID NO: 19) |
| | TCACCCTGCACCAGACTTAA |
| | (SEQ ID NO: 20) |
| | genomic dna adapter oligo |
| Add | AATTGGAGGAGGGAAGGGGG-BIOTIN |
| Biotinylated | |
| Adapter | (SEQ ID NO: 21) |
| | CCTCCTCCCTTCCCCC |
| | (SEQ ID NO: 20) |
| Ligate | genomic dna |
| | GTGGGACGTGGACAGAATTGGAGGAGGGAAGGGGG- |
| | adapter oligo |
| | BIOTIN |
| | (SEQ ID NO: 19) |
| | TCACCCTGCACCTGTCTTAA |
| | genomic dna |

We ligated a biotinylated adapter to the digested genomic sample and purify the ligated material on streptavidin beads and then extend from the adapter to product a range of fragment lengths.

### Polymerase Processivity Instead of Sonication

We have discovered that the natural disassociation of the polymerase from the DNA yields an appropriate distribution of labeled product lengths. While sonication yielded an odd shape in the observed intensities on the microarray, the log-intensities produced by the polymerase's processivity are relatively linear and easier to analyze.

Depending on the polymerase and the distance between restriction sites, we can rely on the polymerase alone or produce shorter fragments by including ddNTPs in the extension reaction.

### Bst Instead of Taq Polymerase

In practice, not all polymerases incorporate ddNTPs with any appreciable frequency. For example, ExTaq generates substantial amounts of product but basically ignores ddNTPs. We have experimented with Bst polymerase as it should incorporate ddNTPs and allow us to control the fragment lengths.

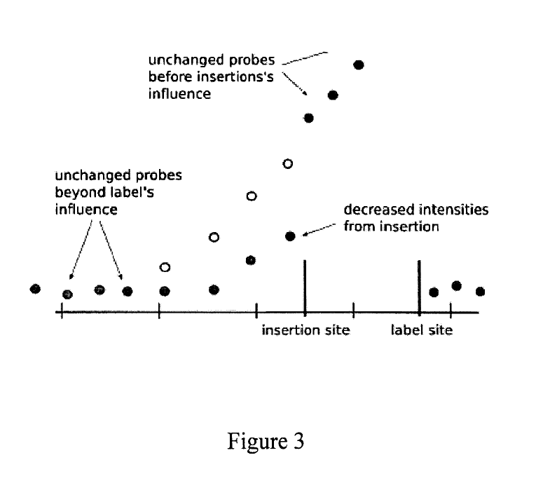

Bst also seems less sensitive to template sequence features that caused ExTaq to reliably terminate the extension. AT and ATT repeats reliably cause ExTaq to terminate an extension, yielding false positive (or at least confounding) signals in the data. Preliminary data, shown in FIG. 3 indicates that Bst successfully copies these regions.

### ULS Labeling

In some cases, the ruler array protocol includes incorporation of Cy-dUTP by polymerase. To address the challenge of analyzing at the AT and ATT repeats, we experimented with other labeling techniques to determine whether repeated incorporation of labeled nucleotides might have caused the termination. One such technique is Universal Linkage System (ULS) labeling that attaches a dye to any nucleic acid strand. This allows us to use plain dNTPs in the extension to avoid sequence bias.

### Example 2

### Distance Analysis

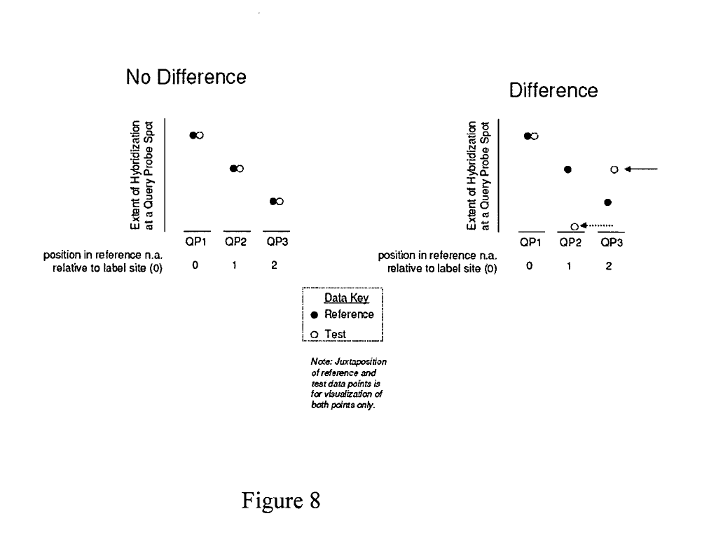

The extent of hybridization (EOH) at a query probe is dependent on distance from the label site (QP1, 0). At the label site this EOH is maximal and it decreases with distance from the site. The reference nucleic acid (na) defines the relationship between extent of hybridization and distance. In the “no difference” panel reference and test na's give the same characteristic decrease in EOH from the label site, indicating no difference. (See FIG. 8)

In the “difference” panel above both test and reference na's exhibit equivalent EOH at the site of label, indicating that this sequence is equivalently present in both na's. The broken arrow indicates a site at QP2 in the test na that has undetectable EOH. Excluding trivial technical reasons, this suggests that this portion of the test na is sufficiently far from the label site so as not to be labeled. It could be that this portion is completely missing from the test na (a deletion) or that it is elsewhere in the test na (a rearrangement) that does not get labeled. A higher level analysis and additional insight (more data) would be required to sort this out. For now assume that some portion of the test na that includes QP2 is missing between position 0 and position 2 relative to the reference na and turn to the broken arrow. Here the test na corresponding to QP3 exhibits an EOH that is consistent with the EOH exhibited by the reference na at QP2, which represents one distance unit from the label site. Combining the insight gained from the information denoted by the solid and broken arrows one can infer that the distance from the label site, QP1 to sequence corresponding to QP3 in the test na is 1 unit versus 2 units as for the reference na. A step further this suggests that the deletion had a size of one unit.

### Example 3

### Ratio Data

### Determining Distances from Ratio Data

Insertions: To determine the size of the insertion strictly from the ratios determine the expected shape in intensities, compute the expected ratio shape for insertions of different sizes and identify the best match for the observed data.

Deletions: The size of the low-ratio region (this is the region in which the probes in one channel give very low intensities because they've been deleted in that genome) is roughly the size of the deletion. Small deletions that do not delete any probes have the same problem as insertions

Inversions: the number of probes at which the ratio is not roughly one gives the size of the inversion. This is particularly easy to detect because the signal will be detected by probes on the opposite strand from the adjacent signal (the material being detected is the reverse complement of what was expected, so the probes design against the other strand will detect it).

### 2) Alternatives to Hidden Markov Models (HMM) for Ratio Data

Pattern recognition methods, also referred to as pattern matching methods, are well known to one of ordinary skill in the art. A number of methods from speech or vision processing that do “pattern matching” against a series of continuous measurements taken over space or time can be used to assess ratio data. For the ruler analysis, there are a few shapes (e.g., insertion, deletion, inversion) that the algorithm serves to match against the observed ratios.

### 3) Analyzing Inversions:

Single-channel (intensity) analysis: looking for inversions is fundamentally the same as looking for insertions or deletions. The problem is still one in which we try to assign a position to each probe such that the observed intensities match the expected intensities. When looking for insertions or deletions, the algorithm moves the probes around but has to keep them in the same order. When looking for inversions, the algorithm can reverse the order for a set of consecutive probes. This increases the running time of the algorithm since there are more possible arrangements of probes it must check, but does not fundamentally change the problem.

Ratio data: inversions have a characteristic signature in ratio data (FIG. 28) that is similar to a deletion but different in that the ratio is constantly changing.

### Example 4

### Genomic Comparison of Two Yeast Strains (Distance Analysis)

The sigma strain of S. cerevisiae has been sequenced at ˜7.5× coverage permitting us to use genomic DNA from Sigma and S288c to assess genomic insertions and deletions using ruler arrays. We performed a genomic comparison two strains of Saccharomyces cerevisiae using the Digest/Ligate/Sonicate protocol. We analyzed the results by plotting in red dots intensities from Σ1278B and in green dots intensities from S288C. We identified location of EcoRI digest sites in the sequences. The intensities in the two channels are very similar close to the EcoRI site. The Σ intensities drop off gradually (the slope extends only in one direction because this microarray only included probes on one strand. An array with probes on both strands would show a symmetric shape) while the S288C intensities drop rapidly at one point. This rapid drop indicates an insertion in S288C relative to Σ. Our in-del detection method detects this sudden change in slope to recognize the insertion.

### Example 5

### Use of Ruler Arrays in Genome Assembly Quality Control

Assembly programs that turn paired-end reads into scaffolds and chromosomes rely on prior knowledge about the distance between the two paired ends. If that expectation about the distance between the two reads is wrong, it may lead to assembly errors. For example, an assembler might erroneously insert space (typically shown in the assembly output as a long string of Ns) not actually present in the genome. Ruler arrays detect such errors in assemblies. We used ruler arrays for the assessment and verification of Σ1278B genome assembly.

### Example 6

### Genomic Comparison of Two Yeast Strains (Ratio Analysis)

In another example of an insertion between two yeast strains, we plot the ratio of the intensities (S288C vs. Σ1278B) at each probe. The sudden drop in ratio from roughly one to a much smaller value (it would be a sudden increase if the channels were swapped) indicates the presence of an insertion. The ratio remains low to the edge of the probes influenced by the restriction site and then returns to roughly one as both the probe observes only background noise in both channels.

In ratio analyses, probes between an insertion/deletion (indel) and the label site yield a ratio of roughly one since these probes are the same distance from the label site in both samples. Probes beyond the indel site yield ratios significantly above or below one since the intensities in one channel will be higher than the intensities in the channel whose probes are now farther away.

The Hidden Markov Model is applied to ratio analyses to explain the data as coming either from the background model (ratio=1) or an insertion (ratio>1 or <1). The HMM assumes that transitions between states are infrequent, so it will not assign single (or even a small number) of high/low ratio probes to the indel state. Tuning the probability of a state change tunes the sensitivity to noise and therefore to small indels. Since the HMM tends to assign the same state to many consecutive probes, the transition from the indel state to the background state give the position of the indel event.

### Example 7

### Learning Expected Intensity Vs Distance Relation

We learned the expected intensity vs distance relation by averaging over all known labeling sites in a dataset. Even if in some of the examples there is an indel or the labeling site has been added or removed, the learned relation is correct. We then compare observed intensities to expected intensities. An insertion will cause lower intensities at probes beyond the insertion site. For each of these probes, we can determine a “shift” (change in genomic coordinates relative to the label site) that would cause the observed intensity to match the expected intensity. Requiring that all probes shift by the same amount makes the analysis more resistant to noise. A single insertion would shift all probes by the same distance, but noisy data may shift different probes by different amounts or in different directions.

### Example 8

### Comparison of Distance Versus Ratio Analyses

Where no indels are present the distance analysis has intensity spikes at the label site that gradually fall off (in one direction if the array observes material from one strand or both directions if the array observes material from both strands). The ratios are one when no indels are present.

An small insertion moves probes farther from the labeling site, so the intensities in the test channel are lower than the intensities at the same probes in the control channel. The ratios are greater than one at probes beyond the insertion site. A larger insertion yields higher ratios since intensities in the test channel are even lower than in the previous small insertion example.

A deletion yields two regions in which the ratio is not one. Probes that have been deleted in the test sample yield a very high ratio. Probes beyond the deleted region yield a ratio<1 since the probe in the test channel is closer to the label site (genomic sequence between it and the label site has been removed). The length of the high-ratio region gives the size of the deletion.

An inversion yields a characteristic zig-zag shape in the distances and ratios since a set of probes have been reordered in one channel relative to the other channel.

### Example 9

### Nick Displacement Through at or ATT Repeats

We developed a labeling strategy that uses a nicking enzyme, such as BsmI and a polymerase that can initiate from the nick and that has a strong strand displacement ability, such as Bst. We performed two ruler array experiments using this methods. At the top are chromosomal coordinates marked in units of 1000 bp. We analyzed blocks marking AT or ATT repeats. Ruler data showed intensities fall off, as expect. However, there was a substantial discontinuity under the ATT repeat when Bst was not used. The second ruler experiment showed data from the Bst polymerase. While the intensities were lower (and noisier since this experiment was done on a reused microarray), there did not seem to be a discontinuity in the intensities, indicating that Bst successfully copies through the ATT repeat.

### Example 10

### Assessment of Distance Based on Frequency of Occurrence of Nucleic Acid Fragments

In the case that:
the frequency of a fragment of length l in the sample population is p(l).
the fragments are labeled throughout such that the total intensity of a fragment increases linearly with its length.
the array probes are spread roughly uniformly through the genome such that the number of probes to which a fragment may bind increases linearly with its length.
The expected intensity at a probe is the sum of the intensities of all fragments bound at that probe

Since a fragment's intensity increases with its length but the number of probes to which it can bind also increases with its length, these two effects cancel and the intensity contributed by probes of length l at some probe is just p(l). Thus, the expected intensity at a probe that is d base pairs from the predefined location is the sum from d to D (the maximum fragment length) of p(l):

$${\sum }_{l = d}^{D} p ( l )$$

If the polymerase terminates the extension with equal probability k at each step, then p(l) will be an exponential distribution:

p(l)=k*(1−k)^{l-1}

such that the intensity at distance d is

$${\sum }_{l = d}^{D} k * {( 1 - k )}^{l - 1}$$

When viewed on a log-scale, this is roughly linear. This gives the relative intensity along an interval between predefined sites; the actual intensity will depend on the number of fragments, the density of the microarray probes, and the density of labeling.

### Example 11

### Ruler-Sequencing (Ruler-Seq) Method

Ruler-seq aim to use short sequencing reads, for example, sequences produced by a Solexa machine (or similar) to screen for insertions and deletions. Virtual array intensities are produced from these Solexa sequencing of the extension product.

Sequence the extension products that we would have hybridized to the microarray. Adapters are and corresponding primers are designed and produced for use in the Solexa sequencing protocol. Using extension products we sequence the ends of fragments farthest from the restriction site. By extending the read back to the restriction site, we can generate virtual array intensities.

### Example 12

### Automated Insertion-Deletion (Indel) Detection Methods

The computational algorithm for detecting indels in two-color Ruler Array experiments simultaneously fits line segments to both channels' log-intensities, attempting to match the segmental boundaries in both channels. The resulting segment boundaries are either restriction sites or represent the boundary of an insertion, deletion, or inversion.

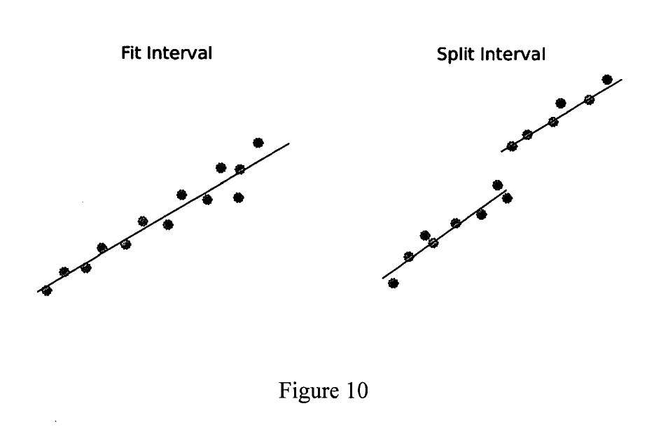

To segment a set of intensity observations (FIG. 10), the algorithm compares two choices:

1. Fit all of the probes with a single line segment.

2. Split the observations at an optimal point and recursively handle each side.

The best split point is found by exhaustively trying all splits. FIG. 8 depicts an example of fitting observations in an interval to either a single segment or two segments.

We used dynamic programming to implement the observation segmentation procedure efficiently on large datasets. When presented with n probe observations along a chromosome, the algorithm constructs a 2D table wherein the row is the interval start probe and the column is the interval end probe. The algorithm first handles the trivial cases such as single points or pairs of points that can be fit with a line. The algorithm then moves on to progressively larger intervals. For the interval [a, b] step #2 above (finding the optimal split) is then easy because the results for all of the intervals [a, k] and [k+1, b] have already been computed. The table used by the dynamic programming algorithm to fit line segments to Ruler Array data typically comprises numbers showing the order in which the algorithm processes subsets of the data.

The algorithm we employ handles both channels simultaneously. For each genomic interval, the algorithm determines which case is most likely given the algorithm's noise model for the data and prior probabilities on the different cases. The algorithm chooses one of four cases for each interval:

1. Fit both channels with lines of the same slope

2. Fit a different line to each channel

3. Fit one channel with a line but split the interval in the other channel

4. Split the interval in both channels

Likely indels are segment boundaries that appear in one channel but not the other. We have implemented our to detect a 100 bp insertion in Σ1278b. Using our dynamic programming algorithm we detected the a 100 bp insertion in Σ1278b, which emerges as a break in the Σ1278b line segment at the insertion site.

To estimate a ruler array's false negative rate, we collected 39 confirmed indels of greater than 100 bp across several chromosomes and attempted to find them with a single ruler array replicate using EcoRI. We recovered 25 of the 39 while generating roughly 100 genome-wide false positive calls and identifying a number of indels smaller than 100 bp (Table 1). Furthermore, at least half of the undetected indels should be found by a ruler array experiment with a different restriction enzyme (this experiment missed them because they were too close to the restriction site). Thus we estimate this would improve the detection of events to over 32 of 39. Our computational framework is able to merge data from multiple ruler array experiments that utilized different enzymes, enabling detection of these events in a single framework.

TABLE 1
Ruler Array false negative rates (genome wide tests)

| False | Confirmed > 100bp Indels | Ruler array | False |
|---|---|---|---|
| Negative Rates | vs. S288C | predicted | negatives |
| Ruler array | 39 | 25 | 36% |
| Ruler array | 39 | >32 | <18% |
| (2 enzymes | | | |
| estimated) | | | |

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Moreover, this invention is not limited in its application to the details of construction and the arrangement of components set forth in the disclosed description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A method for measuring the distance between locations in a nucleic acid, wherein the locations are a predefined location and a test location, comprising:
(a) preparing nucleic acid fragments from the nucleic acid, wherein each fragment comprises (i) only one predefined region, wherein the predefined region is complementary to a predefined location of the nucleic acid and (ii) at least one test region, wherein a test region is complementary to a test location of the nucleic acid, and
(b) measuring the frequency of occurrence of each test region in the nucleic acid fragments, wherein the frequency of occurrence of a particular test region is inversely related to the distance between the test location in the nucleic acid that is complementary to the particular test region and the predefined location in the nucleic acid.

2. The method of claim 1, wherein measuring comprises:
contacting nucleic acid fragments prepared in (a) with at least one polynucleotide under conditions appropriate for hybridization of nucleic acid fragments with the at least one polynucleotide, wherein each polynucleotide is complementary to a test region, and
assessing hybridization of nucleic acid fragments with the at least one polynucleotide, wherein the extent of hybridization is indicative of the frequency of occurrence of the test region complementary to the at least one polynucleotide.

3. The method of claim 1, wherein the measuring comprises
sequencing nucleic acid fragments prepared in (a) to obtain fragment sequences and
assessing the occurrence of each test region in the fragment sequences to obtain the frequency of occurrence of each test region in the nucleic acid fragments.

4. A method for detecting an aberration in a nucleic acid comprising determining a distance between two locations in the nucleic acid by the method of any of claim 1, and comparing the distance to a reference distance, wherein the result of the comparison is indicative of the aberration.

5. The method of claim 4, wherein the aberration is an inversion, insertion, or deletion.

6. The method of claim 1, wherein the predefined location is a restriction site.

7. The method of claim 6, wherein preparing comprises digesting the nucleic acid with a restriction enzyme to produce restriction fragments.

8. The method of claim 7, further comprising ligating an adapter to the restriction fragment ends to produce adapter ligated restriction fragments.

9. (canceled)

10. The method of claim 1, wherein preparing comprises performing a extension reaction on the nucleic acid to produce the nucleic acid fragments, wherein the reaction includes a polymerase, a primer complementary the predefined location, a reaction buffer, and a nucleotide mixture.

11. The method of claim 10, wherein the nucleotide mixture comprises one or more dideoxynucleotides.

12. The method of claim 10, wherein the nucleotide mixture comprises one or more labeled nucleotides.

13. (canceled)

14. (canceled)

15. The method of claim 12, further comprising separating labeled nucleic acid fragments.

16. The method of claim 1, wherein the preparing comprises incorporating a biotin moiety in nucleic acid fragments.

17. The method of claim 15, wherein the nucleic acid fragments are separated by contacting the biotin moiety with streptavidin that is fixed to a solid support under conditions that result in binding of biotin moieties to the streptavidin.

18. The method of claim 1, wherein preparing comprises sonicating the nucleic acid.

19. The method of claim 1, further comprising labeling the nucleic acid fragments with a universal labeling system (ULS).

20. The method of claim 2, wherein the at least one polynucleotide is fixed to a solid support.

21. The method of claim 20, wherein the at least one polynucleotide is a constituent of a query probe.

22. The method of claim 20, wherein the solid support is an array.

23. The method of claim 22, wherein the array is a genome microarray, chromosome array, or CpG island array.

24. (canceled)

25. (canceled)

26. A method for detecting a difference between a test nucleic acid and a reference nucleic acid comprising:
(a) contacting (i) a collection of labeled test nucleic acid fragments with (ii) a set of query probes, wherein test nucleic acid fragments are labeled at one or more defined site to produce the labeled test nucleic acid fragments and wherein a query probe is a polynucleotide and the set of query probes comprises at least three different polynucleotides, each of whose sequence identifies a known region in the reference nucleic acid, under conditions appropriate for hybridization of labeled test nucleic acid fragments with query probes;
(b) determining the extent of hybridization between each query probe and labeled test nucleic acid fragments;
(c) associating the extent of hybridization for each query probe, characteristic(s) of the known region identified by the query probe, and characteristic(s) of the defined sites, thereby producing a test hybridization pattern;
(d) determining distance in the test hybridization pattern by evaluating the extent of hybridization for a query probe within the resolution limit of a defined site within the test hybridization pattern with (i) the extent of hybridization of the query probe in a reference hybridization pattern and (ii) distance from the query probe to the defined site in the reference hybridization pattern, wherein distance is the number of bases between a defined site and a region identified by a query probe; and
(e) identifying a difference in distance between the reference hybridization pattern and the test hybridization pattern, thereby detecting a difference between a test nucleic acid and a reference nucleic acid.

27. A method for detecting a difference between a test nucleic acid and a reference nucleic acid comprising:
(a) contacting (i) a collection of labeled test nucleic acid fragments with (ii) a set of query probes, wherein test nucleic acid fragments are labeled at one or more defined sites to produce the labeled test nucleic acid fragments and wherein a query probe is a polynucleotide and the set of query probes comprises at least three different polynucleotides, each of whose sequence identifies a known region in the reference nucleic acid, under conditions appropriate for hybridization of labeled test nucleic acid fragments with query probes;
(b) determining the extent of hybridization between each query probe and labeled test nucleic acid fragments;
(c) associating the extent of hybridization for each query probe, characteristic(s) of the known region identified by the query probe, and characteristic(s) of the defined sites, thereby producing a test hybridization pattern;
(d) comparing the test hybridization pattern with a reference hybridization pattern to produce a ratio hybridization pattern; and
(e) identifying a significant local maximum or a significant local minimum in the ratio hybridization pattern,
thereby detecting a difference between a test nucleic acid and a reference nucleic acid.

28. The method of claim 26, wherein the lengths of the test and reference nucleic acid fragments is have a random distribution.

29. The method of claim 28, wherein the random distribution of test nucleic acid fragments is substantially equivalent to the random distribution of reference nucleic acid fragments.

30. The method of claim 26, wherein the majority of fragments are from about 3-kb to about 5-kb.

31. The method of claim 26, wherein defined sites are defined by the sequence specificity of one or more restriction enzymes.

32. The method of claim 31, wherein one of the one or more restriction enzymes is Ecortl.

33. The method of claim 31, wherein one of the one or more restriction enzymes is BamHI.

34. The method of claim 31, wherein at least one of the one or more restriction enzymes is methylation sensitive.

35. The method of 31, further comprising contacting labeled nucleic acid fragments with the one or more restriction enzymes under conditions suitable for digestion of the nucleic acid fragments by the one or more restriction enzymes at defined sites, thereby producing digested labeled nucleic acid fragments.

36. The method of claim 35, further comprising ligating an adapter to digested nucleic acid fragments to produce linker-ligated nucleic acid fragments.

37. The method of claim 36, wherein the adapter comprises at least one detectable nucleotide.

38. The method of claim 36, further comprising linear PCR with the linker-ligated nucleic acid fragments as a template to produce the labeled nucleic acid fragments, wherein the linear PCR is primed by a primer comprising a sequence complementary to a portion of the adapter.

39. The method of claim 26, wherein the defined sites are specified by 10 one or more PCR primers, wherein the PCR primers are used to prime a linear PCR reaction with the nucleic acid fragments as a template.

40. The method of claim 39, wherein the linear PCR incorporates a detectable nucleotide, thereby producing the labeled nucleic acid fragments.

41. The method of claim 40, wherein the detectable nucleotide is a fluorophore-conjugated nucleotide.

42. The method of claim 41, wherein the fluorophore has an excitation peak of about 492 nm and emission peak of about 510 nm, an excitation peak of about 550 rim and emission peak of about 570 rim, or an excitation peak of about 650 rim and emission peak of about 670 rim.

43. The method of claim 41, wherein the fluorophore is Cy3 or Cy5.

44. The method of claim 26, wherein the query probes are arranged in an array.

45. The method of claim 44, wherein the array is a genomic microarray, a 5 chromosome array or, a CpG island array.

46. A method for labeling DNA, comprising:
(a) combining:
(i) linear DNA that comprises DNA to be labeled and adapter 10 DNA that tags each end of the DNA to be labeled, wherein the adapter DNA flanks the DNA to be labeled.
(ii) primer capable of hybridizing to the adapter DNA; and
(iii) labeled nucleotides or combining:
(i) linear DNA to be labeled
(ii) a primer capable of hybridizing to a specific sequence in the linear DNA; and
(iii) labeled nucleotides, thereby producing a combination; and
(b) maintaining the combination under conditions appropriate for amplification of the linear DNA to occur, thereby producing amplified DNA comprising at least one labeled nucleotide, thereby producing labeled DNA.

47. A method of producing a pool of labeled DNA fragments, wherein the pool comprises a random distribution of labeled DNA fragments of from about 3 kilobases to about 5 kilobases, comprising:
(a) combining:
(i) linear DNA that comprises DNA to be labeled and adapter DNA that tags each end of the DNA to be labeled, wherein the adapter DNA flanks the DNA to be labeled.
(ii) primer capable of hybridizing to the adapter DNA; and
(iii) labeled nucleotides
or combining:
(i) linear DNA to be labeled
(ii) a primer capable of hybridizing to a specific sequence in the linear DNA; and
(iii) labeled nucleotides, thereby producing a combination; and
(b) maintaining the combination under conditions appropriate for amplification of the linear DNA to occur, thereby producing amplified DNA comprising at least one labeled nucleotide, thereby producing a pool of labeled DNA fragments.
